(19) United States
(12) Patent Application Publication
(10) Pub. No.: US 2026/0224237 A1
(43) Pub. Date: Aug. 6, 2026

(54) HIGH ACOUSTIC ENERGY CATHETER SYSTEM FOR HEART VALVE CALCIUM MODIFICATION

(71) Applicant: Cardiovascular Systems, Inc., St. Paul, MN (US)

(72) Inventors: Ethan C. Korngold, Portland, OR (US); William G. Besser, St. Paul, MN (US); Daniel J. Vreeman, Otsego, MN (US); Matthew W. Tilstra, Rogers, MN (US); Gregory B. Ingersoll, Minneapolis, MN (US); Gregory K. Olson, Elk River, MN (US)

(21) Appl. No.: 19/531,964

(22) Filed: Feb. 6, 2026

Related U.S. Application Data
(60) Provisional application No. 63/904,986, filed on Oct. 24, 2025.
(60) Provisional application No. 63/816,785, filed on Jun. 3, 2025.
(60) Provisional application No. 63/754,970, filed on Feb. 6, 2025.

(51) Int. Cl. A61B 17/22; A61B 17/00

(52) U.S. Cl. CPC A61B 17/22022; A61B 17/00234; A61B 2017/00172; A61B 2017/00199; A61B 2017/00243; A61B 2017/00305; A61B 2017/00367; A61B 2017/00544; A61B 2017/00557; A61B 2017/22025; A61B 2017/22065

(57) ABSTRACT

An elongate catheter for providing high energy acoustic waves to a heart valve region of a subject, the catheter system including: an inflatable balloon at a distal portion of the catheter system, the inflatable balloon adapted to traverse a vessel of the subject in a radial retracted configuration to reach a treatment site in the heart valve region and receive fluid to inflate to an expanded configuration at the treatment site; a deflectable distal tip portion distal to the inflatable balloon; an emitter within the inflatable balloon; a conductive pathway for supplying energy to the emitter to produce a spark to generate high energy acoustic waves within fluid inflating the balloon; and a movable control member operatively connected to the distal portion for deflecting the distal tip portion in a direction away from the longitudinal axis of the catheter system.

### CROSS-REFERENCE TO RELATED APPLICATIONS

This application claims priority to and the benefit of U.S. Provisional Application No. 63,754,970, filed February 6, 2025, and entitled HIGH ACOUSTIC ENERGY CATHETER SYSTEM FOR HEART VALVE CALCIUM MODIFICATION, and U.S. Provisional Application No. 63/816,785, filed June 3, 2025, and entitled HIGH ACOUSTIC ENERGY CATHETER SYSTEM FOR HEART VALVE CALCIUM MODIFICATION, U.S. Provisional Application No. 63/904,986, filed October 24, 2025, and entitled HIGH ACOUSTIC ENERGY CATHETER SYSTEM FOR HEART VALVE CALCIUM MODIFICATION, the entire contents of which are incorporated herein by reference in their entireties.

### TECHNICAL FIELD

The present disclosure is directed to a catheter system for treating a calcified heart valve.

### BACKGROUND

The four chambers of a heart 10 (see FIG. 1) include two upper chambers that collect blood flowing into the heart and two lower chambers that pump blood out of the heart to the lungs or other parts of the body. Each chamber has a corresponding valve. Each valve has a set of flaps or leaflets which act as one-way inlets, when open, for letting blood out of the corresponding chamber of the heart (e.g., for letting blood flow through the valve) and when closed, for preventing backflow.

A tricuspid valve 12 is located between a right atrium 14 and a right ventricle 16 to allow blood to flow from the right atrium 14 into the right ventricle 16. A pulmonary valve 18 is located between the right ventricle 16 and a pulmonary artery 20 and regulates flow of oxygen-poor blood from the heart to the lungs. A mitral valve 22 (also known as a bicuspid or left atrioventricular valve) is located between a left atrium 24 and a left ventricle 26 to allow blood to flow from the left atrium 24 into the left ventricle 26. Lastly, an aortic valve 28 is located between the left ventricle 26 and an aorta 30, which is the largest blood vessel in the body for delivering oxygenated blood from the heart to the rest of the body. The aortic valve 28 opens to allow blood flow from the left ventricle 26 to the aorta 30.

FIG. 2 is a top view showing the four valves 12, 18, 22 and 28. The mitral valve 22 has two leaflets (or flaps) 32a and 32b. Each of the remaining valves has three leaflets (or flaps) – specifically, the tricuspid valve 12 has leaflets 34a, 34b and 34c; the pulmonary valve 18 has leaflets 36a, 36b and 36c; and the aortic valve 28 has leaflets 38a, 38b and 38c.

Over time, a heart valve can become calcified, meaning that a large amount of calcium can be deposited on or around the valve. A calcified heart valve can become stiff and narrowed (stenotic), thereby resulting in increased resistance of blood flowing through the heart and overall increased load on the heart. More commonly, it is the left sided valves that calcify, relative to the valves on the right side of the heart. FIG. 3 shows the left side of the heart and more specifically, shows an uncalcified aortic valve 28 in an open position such that there is a sufficient opening for blood to flow from the left ventricle 26 into the aorta 30. By contrast, FIG. 4 shows a calcified aortic valve 28’ in an open position. Due to calcium lesions (or calcifications) 40 on and surrounding the leaflets of the aortic valve 28’, the valve 28’, when open, has a significantly narrower opening, compared to an uncalcified valve. This narrowing can lead to symptoms ranging from mild to severe, including chest pain, shortness of breath, fatigue etc.

The most common valve in patients needing repair is the aortic valve. Depending on the extent of the calcification, the aortic valve may be replaced with a synthetic valve, a tissue valve or other alternatives. For many patients, repair of the valve, particularly if non-invasive, may be preferable to valve replacement.

### BRIEF DESCRIPTION OF THE DRAWINGS

These drawings are exemplary illustrations of certain embodiments and, as such, are not intended to limit the disclosure.

FIG. 1 is a schematic of a heart showing the chambers, valves and direction of blood flow.

FIG. 2 is a top view schematic of the heart of FIG. 1 showing the four heart valves.

FIG. 3 is a schematic of the left side of the heart of FIG. 1 showing the aortic valve in an open position.

FIG. 4 is a schematic of the left side of the heart showing a calcified aortic valve in an open position.

FIG. 5 illustrates a heart valve catheter system for performing high energy acoustic lithotripsy of a heart valve region of a patient, according to one or more embodiments of the present disclosure.

FIG. 6 is a front view of a portion of the heart valve catheter system of FIG. 5 in an inflated state inside the calcified aortic valve of FIG. 4 for providing valve repair, according to one or more embodiments of the present disclosure.

FIG. 7 is a front view of another example of a portion of a heart valve catheter system for performing high energy acoustic lithotripsy, inserted into a partial cross-sectional view of a heart, according to one or more embodiments of the present disclosure.

FIG. 8 is a front view of another example of a portion of a heart valve catheter system for performing high energy acoustic lithotripsy of a heart valve region, inserted into a partial cross-sectional view of a heart, according to one or more embodiments of the present disclosure.

FIG. 9 is a front view of a portion of the heart valve catheter system of FIG. 8 for performing high energy acoustic lithotripsy, according to one or more embodiments of the present disclosure.

FIG. 10 is a front view of a portion of the heart valve catheter system of FIG. 8 for performing high energy acoustic lithotripsy of a heart valve region according to one or more embodiments of the present disclosure.

FIG. 11 is a front view of a portion of the heart valve catheter system of FIG. 8 for performing high energy acoustic lithotripsy of a heart valve region according to one or more embodiments of the present disclosure.

FIG. 12 is a front view of a portion of the heart valve catheter system of FIG. 8 for performing high energy acoustic lithotripsy of a heart valve region according to one or more embodiments of the present disclosure.

FIG. 13 is a cross-sectional view of the heart valve catheter system of FIG. 8 for performing high energy acoustic lithotripsy of a heart valve region as viewed from a proximal end of the system toward a distal end of the system, according to one or more embodiments of the present disclosure.

FIG. 14 is a cross-sectional view of the heart valve catheter system of FIG. 8 for performing high energy acoustic lithotripsy of a heart valve region as viewed from a proximal end of the system toward a distal end of the system, according to one or more embodiments of the present disclosure.

FIG. 15 is a process flow chart of a method of reducing calcifications on a heart valve, according to one or more embodiments of the present disclosure.

FIG. 16 is a front view of another example of a heart valve catheter system for performing high energy acoustic lithotripsy, inserted into a partial cross-sectional view of a heart, according to one or more embodiments of the present disclosure.

FIG. 17 is a front view of the heart valve catheter system for performing high energy acoustic lithotripsy of FIG. 16, in a different position within a partial cross-sectional view of a heart, according to one or more embodiments of the present disclosure.

FIG. 18 is a front view of the heart valve catheter system for performing high energy acoustic lithotripsy of FIG. 16, in a different position within a partial cross-sectional view of a heart, according to one or more embodiments of the present disclosure.

FIG. 19 is a front view of a heart valve catheter system for performing high energy acoustic lithotripsy of a heart valve region of a patient, according to one or more embodiments of the present disclosure.

FIG. 20 is a front view of a portion of a heart valve catheter system for performing high energy acoustic lithotripsy of a heart valve region of a patient, according to one or more embodiments of the present disclosure.

FIG. 21 is an enlarged front view of a portion of FIG. 20.

FIG. 22 is an enlarged perspective view of a portion of FIG. 21.

FIG. 23A is a perspective view of a distal tip portion of a heart valve catheter system with a flex tip, in accordance with an embodiment of the present disclosure.

FIG. 23B is a perspective view of a distal tip portion of a heart valve catheter system with a flex tip, in accordance with an embodiment of the present disclosure.

FIG. 23C is a perspective view of a distal tip portion of a heart valve catheter system with a flex tip, in accordance with an embodiment of the present disclosure.

FIG. 23D is a perspective view of a distal tip portion of a heart valve catheter system with a flex tip, in accordance with an embodiment of the present disclosure.

FIG. 23E is a perspective view of a distal tip portion of a heart valve catheter system with a flex tip, in accordance with an embodiment of the present disclosure.

FIG. 23F is a perspective view of a distal tip portion of a heart valve catheter system with a flex tip, in accordance with an embodiment of the present disclosure.

FIG. 24A is a perspective view of a distal tip portion of a heart valve catheter system with a flex tip, in accordance with an embodiment of the present disclosure.

FIG. 24B is a top view of a stenotic aortic heart valve.

FIG. 24C is a top view of a stenotic aortic heart valve.

FIG. 25A is a perspective view of a distal tip portion of a heart valve catheter system with a flex tip, in accordance with an embodiment of the present disclosure.

FIG. 25B is a top view of a stenotic aortic heart valve.

FIG. 25C is a top view of a stenotic aortic heart valve.

FIG. 26A is a schematic top view of a heart valve with calcium deposits.

FIG. 26B is a schematic side view of emitters at a distal tip portion of a heart valve catheter system positioned relative to a heart valve having areas of calcium deposits.

FIG. 27 is a schematic side view of a steerable catheter for use as part of a heart valve catheter system.

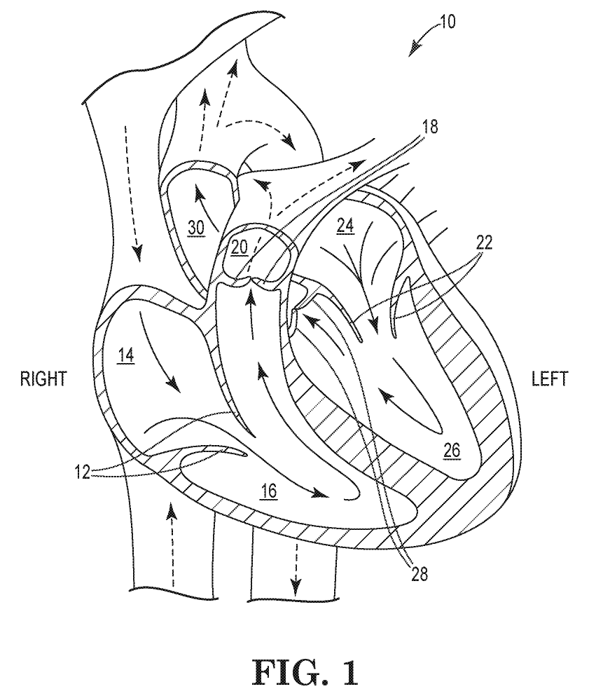

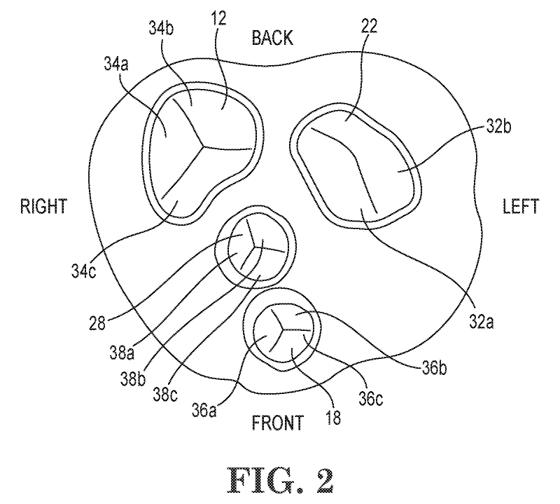

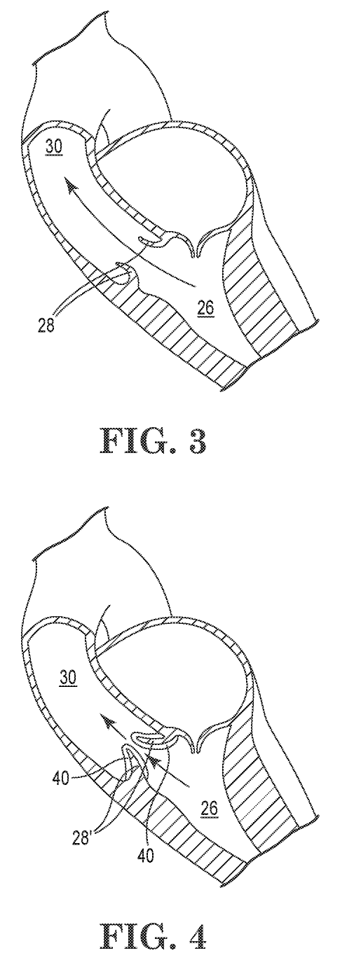

### DETAILED DESCRIPTION

The present disclosure is directed to a catheter system for treating a calcified heart valve. More specifically, the present disclosure is directed to a heart valve catheter system that includes a deflectable distal tip to aid in placement of the catheter into a desired position within a chamber of the heart. Such a catheter system does not require the use of a guide wire, but certain embodiments will utilize a guide wire.

The heart valve catheter system includes an inflatable balloon that can be inflated with a conductive medium, such as saline, and one or more emitters. Each emitter comprises a pair of spaced electrodes, with at least one emitter electrically connected to a high voltage pulse generator. Such a high voltage pulse generator preferably can create one or a series of high voltage pulses of 2000 volts or more, for example. When a high voltage pulse is generated, a spark is created across the one or more pairs of electrodes that can be connected in series or parallel through the conductive medium within the balloon to perform high energy acoustic lithotripsy of a heart valve region. In accordance with one aspect of the present invention, the spark in the inflated balloon results in an energy wave for breaking up calcifications on or around the calcified heart valve.

The high energy acoustic lithotripsy of a heart valve region catheter system described herein can be used as a less invasive alternative to valve replacement. It may be used to break up calcifications on or around the leaflets of a heart valve. It may be used on any of the four valves of a patient’s heart. It may be used to prepare a heart valve region of a patient for a subsequent transcatheter heart valve replacement procedure. The use of the present invention is believed to reduce the chances of paravalvular leakage after a transcatheter heart valve replacement procedure.

Catheter systems having an angioplasty balloon have been commonly used to apply a physical force by expansion of the balloon against a calcified lesion within vasculature to force the calcification back into and against the blood vessel wall. Some such calcified lesions and thrombi are not effectively broken up by the use of an angioplasty balloon alone. More recently, catheter systems have been developed that include a balloon similar to an angioplasty balloon that is filled with a conductive liquid medium, such as a saline solution, for expanding the balloon in position at the lesion or thrombus, wherein the catheter system includes one or more pairs of electrodes (emitters) operatively positioned within the conductive liquid medium. The electrodes are pulsed with high voltage direct current so as to create a spark that jumps over a gap between the two electrodes of each emitter at each pulse. The spark within the conductive medium creates an energy wave that propagates through the liquid medium and through the wall of the balloon, causing the balloon to physically provide a force against the lesion or thrombus. The energy propagation includes the creation of micro-bubbles that also facilitate the physical force. Such intravascular lithotripsy (IVL) devices can be provided in different designs and sizes, and utilize a reusable power source such as an IVL generator. Examples of IVL devices and systems can be found in the following patent applications: US Patent Application Pub. Nos. 2024/0156478, 2024/0180569, and 2024/0307081; International Publication Numbers WO 2023/015047, WO 2024/138212, and WO 2024/107470, the contents of which are hereby incorporated by reference in their entireties. Examples of power sources or generators or controllers useful for IVL procedures are described in published U.S. Patent Application Nos. 2024/0180569 and 2025/0000532, the contents of which are hereby incorporated by reference in their entireties. The present invention is preferably compatible with or used with an energy source or generator (e.g. a controller) that is also able to provide energy for an IVL procedure and its associated catheters. The present invention may also be used with an energy source or generator or controller provided for procedures in the heart valve region of a patient.

The present disclosure focuses on using high energy acoustic lithotripsy of a heart valve region rather than a vascular application. As an example, one such device comprises a 0.36mm (0.014-inch) guide wire-compatible, fluid-filled balloon catheter with two lithotripsy emitters incorporated into the shaft of a 12-mm-long balloon segment. Other examples of devices include a number of different sized guide wire-compatible, fluid-filled balloon catheters, such as 0.46mm (0.018-inch) or 0.89mm (0.035-inch), for example. A fluid filled balloon (e.g. a 50/50 saline contrast medium) is inflated to 4 atm, or about 4 atm, and then electrical pulses are provided to the emitters that create high voltage sparks to provide the therapy against the lesion. Acoustic waves (also referred to herein as high energy acoustic waves) are created and the calcium is fractured.

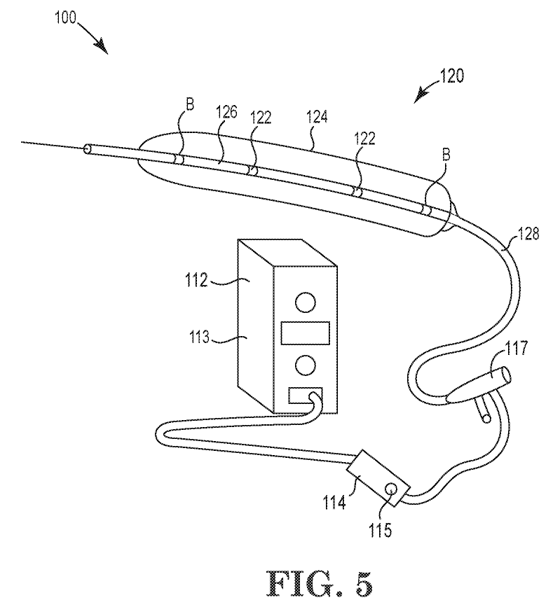

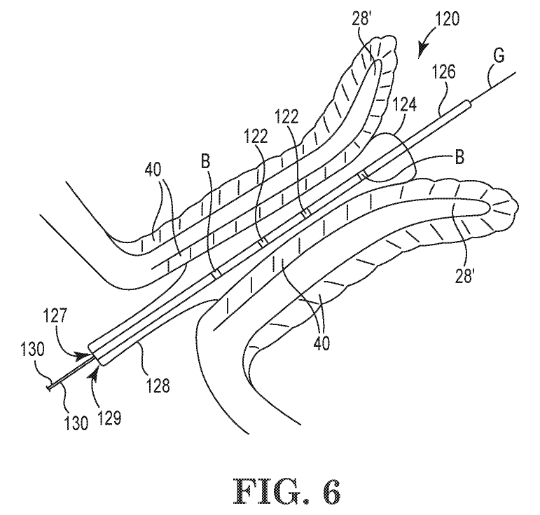

FIG. 5 shows a heart valve catheter system 100 for performing high energy acoustic lithotripsy of a heart valve region according to the present invention and comprising a high voltage power source that is provided as a console 112 (in the form of a high voltage electrical pulse generator, but alternatively in the form of a laser system), a controller 113 (within the console 112), a handle 114 with a therapy delivery switch 115, a catheter 120 having a central tube 126 with two lithotripsy emitters 122, and a balloon 124. FIG. 6 shows the catheter 120 inserted inside the heart, with the balloon 124 in an inflated state in proximity to a calcified aortic valve 28’ of FIG. 4. As shown in FIG. 5, each emitter 122 represents a pair of arcing electrodes, but alternatively they could comprise optical or laser emitters. The controller 113 can be housed in the same unit as the power source 112 (as shown in FIG. 5). In other embodiments, the power source 112 and controller 113 can be in separate units. Optional marker bands B may be provided to afford visualization and proper positioning by use of known imaging techniques. A catheter sheath 128 is connected at its distal end to a proximal end of the balloon 124. A proximal end of the sheath 128 is connected to a hub 117.

The balloon 124 can be filled with a conductive medium in order to inflate the balloon 124 and as such, the catheter 120 can be connectable to a source for providing the conductive fluid or medium. The conductive fluid or medium can be supplied to the balloon 124 by a lumen within the catheter sheath 128 for controlling the balloon inflation and desired pressure therein.

The balloon 124 can be formed of a compliant, semi-complaint, or noncompliant balloon material. In some embodiments, the balloon 124 comprises material that is less than 0.023mm (0.0009 inches) thick in an unstretched state prior to inflation. The material for the balloon 124 can also be referred to as a “thin-walled balloon material.” One such material comprises a polyamide having repeating units linked by amide links. Some Nylon and PEBAX materials are suitable. Such materials may also include materials such as a composite and/or multilayer structure. Compliant materials include silicone, polyurethane and/or nitinol materials. In some embodiments, thin-walled balloon materials may comprise multi-layer balloon designs and the fluids filling the inner balloon may be dissimilar to the fluid filling the outer balloon to exploit differences in electrical insulative properties, speeds of sound of materials, etc. Compliant, semi-compliant, and non-compliant materials may include nylon, polyurethanes, silicones, polyethylene terephthalate (PET) and other biocompatible materials. In one embodiment, a burst pressure of thin-walled balloons is typically between 4 and 20 atm, or between about 4 and 20 atm, more preferably between 8 and 12 atm. In one example, a Nylon 12 material is used with a rated burst pressure of between 9 and 12 atm, or between about 9 and 12 atm.

The central tube 126 preferably defines a guide wire lumen 127 through which a guide wire G passes for delivering the balloon 124 at the desired location along the guide wire G. The sheath 128 preferably surrounds the central tube 126 and defines a delivery lumen 129 through which saline can be controllably delivered for balloon inflation. The lumen 129 can provide a concentric space around the central tube 126 within which electrode wires 130 can be run from the switch 115 to the emitters 122. The hub 117 can include any number of ports allowing the electrode wires 130 to pass into the lumen 129 along with saline for inflation, the guide wire G, and any number of other components as desired.

During insertion into a subject or patient, the balloon 124 may be placed in a deflated position to more readily be advanced through the patient’s vasculature and into the heart and ultimately into the respective heart chamber(s) to arrive in proximity to a region of calcification.

Once in position with the balloon 124 inflated (as shown in FIG. 6), the lithotripsy emitters 122 may be “fired” to disrupt the calcium lesions. While two electrode pair emitters 122 are shown in FIGS. 5 and 6, in other embodiments the number of electrode pair emitters can comprise only one emitter or more than two emitters (3, 4, 5, 6 or more emitters). The terms “electrode pair” and “emitter” or “electrode pair emitter” are used interchangeably in the present disclosure.

A high voltage pulse is provided to one of the two emitters 122 and, in accordance with the illustrated embodiment, then in series to the second of the two emitters 122. The high voltage pulse is a minimum voltage of at least 2000 volts and in some embodiments, a voltage of at least 3000 volts. The high voltage pulse causes a spark across the electrodes in the first emitter 122, then or also across the electrodes in the second emitter 122. The somewhat conductive saline solution within the balloon 124 permits the high voltage spark across each electrode pair, thus creating an energy wave that propagates within the balloon 124 toward the calcification 40, resulting in a force that breaks up the calcification 40.

The controller 113 of FIG. 5 is used to deliver voltage pulses, as described above. The controller 113 can regulate delivery of such voltage pulses in terms of frequency, attaining a desired pressure from the energy wave, and the like.

Control by the controller 113 can be done including manual manipulation by an operator. Alternatively, some or all of the steps of each operation can be automated. The controller 113 can include any number of control modules, such as utilizing electronic switches, or for controlling part or all of sizing and/or high energy acoustic lithotripsy system operation. The controller 113 can also assist in automating the inflation and deflation of the balloon through an internal fluid pump or compressor and pressure gauge, monitored and controlled by programmed electronics. Such a control system can include any number of data processors, memory, and programming provided as software or firmware.

Although a focus herein is on repair of the calcified aortic valve, the present disclosure and heart valve catheter system is applicable for use with any of the heart valves. The balloon 124 can be inflated to a typical angioplasty pressure, such as 4 atm, or it may be inflated to a higher pressure, such as, for example, 6 atm, to aid in engaging the heart valve calcifications. In some embodiments, the pressure may range between 4 and 6 atm, or between about 4 and about 6 atm, as additional pressure may be required, as compared to that commonly used for a vessel wall.

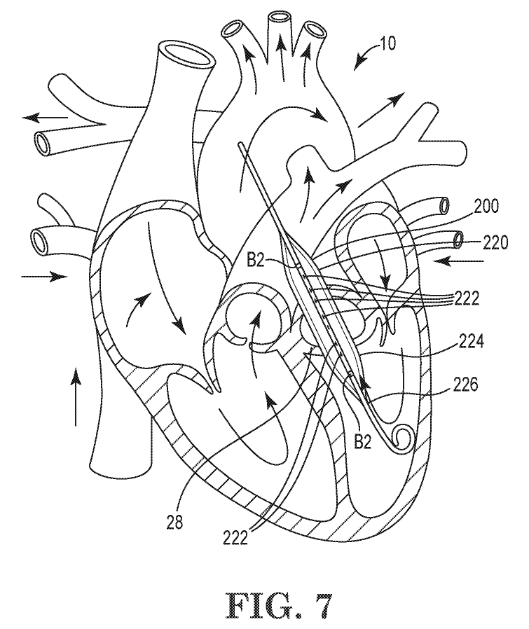

FIG. 7 shows another example of a heart valve catheter system 200 in position inside the heart 10. The heart valve catheter system 200 is similar in design to the system 100 of FIGS. 5 and 6, and includes a catheter 220, a central tube 226 with emitters 222 and marker bands B2, and a balloon 224. This embodiment illustrates a general location or position of where the catheter 220 can be placed in the heart 10 in order to perform procedures on the aortic valve 28. In some embodiments, the system 200 can include an optional atraumatic tip at the distal end of the catheter 220 for passage through the valve 28 and anchoring of the catheter 220 once in position.

In the embodiment of FIG. 7, there are six emitters 222. As provided above, in other embodiments, the catheter 220 can include more or fewer emitters than the six shown in FIG. 7.

The system 200 can include catheters 220 in a range of sizes, including a range of sizes for the balloon 224, such that the system 200 can accommodate variability in patient anatomy and/or differences in size of the ventricles/atriums and heart valve annuli. For example, an annulus of the aortic valve 28 may be larger than an annulus of the mitral valve 22. Thus, a catheter designed for repair of the aortic valve 28 may have a bigger balloon than a catheter designed for repair of the mitral valve.

Given the larger size of the valves 12, 18, 22 and 28 in the heart 10, relative to other vessels in the vascular system, including coronaries and peripheral blood vessels, the balloons for repair of a heart valve may be greater in diameter than blood vessel angioplasty balloons. However, it is contemplated that valve sizes that are not common now could be more common in the future such that the relative sizes of the balloons as compared to the valves can be different. With larger diameter size balloons, more energy may be needed in order to create a higher energy spark for creating energy waves that propagate to the balloon walls (a comparatively greater distance) and ultimately, the lesion or calcification on the heart valve. As such, the high voltage pulse may be greater than the voltages described above, which include a preferred range of 2700V and 3700V, or a preferred range of about 2700V and about 3700V. In an example, the high voltage pulse for heart valve repair can be between 2000V and 5000V, or between about 2000V and about 5000V, and preferably between 3500V and 4500V. Pulses of 6000V or higher are also contemplated. Optionally, a plurality of emitters with less than high voltage pulses may be provided, all or some of which may not be high voltage emitters.

Moreover, the pressure may be greater than the inflation pressures provided above, which include a range from 4 atm to 6 atm, or from about 4 atm to about 6 atm. Alternatively, the pressure can be lower. In embodiments, it is preferred that the pressure causes a balloon inflation to minimally have the outer balloon surface contact the valve leaflets around the balloon so that one or more high energy acoustic waves are effectively transferred to the valve leaflets to break up a lesion. In an example, the inflation pressure for a balloon for heart valve repair can be between 2 atm and 10 atm, or between about 2 atm and about 10 atm. The pressure requirement can depend on factors such as the size and type of the balloon, the specific valve being treated, and the patient’s anatomy. This procedure can be guided by imaging techniques like fluoroscopy to ensure proper placement and inflation of the balloon.

In some embodiments, one or both of the high voltage pulse and the inflation pressure can be greater for a high energy acoustic lithotripsy system than the values used within an intravascular lithotripsy (IVL) system. One or both of the voltage and pressure can be determined based, in part, on a size of the balloon, the size of the particular valve it is to be implanted in, and the size of the lesion or calcification. More specifically, considerations for determining balloon sizing and inflation pressure include the patient’s valve anatomy and size. Accurate measurement of the valve annulus (the ring-like structure where the valve leaflets attach) is desirable. This can be done using imaging techniques such as echocardiography, computed tomography (CT), and/or magnetic resonance imaging (MRI). Patient specific considerations can also include the patient’s overall anatomy, including the size and shape of the heart chambers and the presence of any calcifications or other abnormalities. The specific procedure being performed (high energy acoustic lithotripsy procedures with different numbers of emitters, balloon lengths, number of high voltage pulses, and the like) can also influence the choice of balloon size. Different procedures may require different balloon diameters and lengths. Additionally, the compliance (flexibility) of the balloon affects how it expands within the valve. Non-compliant balloons expand to a fixed size and are used for precise dilations, while compliant balloons can expand more variably and are used for more flexible applications.

A method for performing a medical procedure on a subject to modify (i.e. remove or reduce) calcifications on a heart valve of the subject can begin by positioning the balloon catheter within a calcified heart valve. Then, the balloon can be inflated, engaging the external surface of the balloon with the surrounding calcium, disrupting the calcium. If the operator(s) decides to conduct a further high energy acoustic lithotripsy of a heart valve region treatment, the operator can activate one or more, or a series of voltage pulses to further break up the calcium. Thereafter, the operator(s) can make a determination as to whether there is additional calcium on the heart valve to treat. Such determination can include using fluoroscopy or any available imaging techniques, including echocardiography, CT, MRI, PET and SPECT, for example, to view the heart valve and extent of calcification. In any of these procedures, the firing of emitters for the high energy acoustic lithotripsy treatment may be controlled based upon feedback data of the heartbeat of a subject patient.

It should be understood that, depending on the example, certain acts or events of any of the methods described herein can be performed in a different sequence, may be added, merged, or left out altogether (e.g., not all described acts or events are necessary for the practice of the method). Moreover, in certain examples, acts or events may be performed concurrently (e.g., through multi-threaded processing, interrupt processing, and/or multiple processors) rather than sequentially. In addition, while certain aspects of this disclosure are described as being performed by a single circuit or unit for purposes of clarity, it should be understood that the techniques of this disclosure may be performed by a combination of units or circuits associated with, for example, a medical device.

In one or more examples, the functions described may be implemented in hardware, software, firmware, or any combination thereof. If implemented in software, the functions may be stored as one or more instructions or code on a computer-readable medium or memory and executed by a hardware-based processing unit or microprocessor. Computer-readable media may include computer-readable storage media or memory, which corresponds to a tangible medium such as data storage media (e.g., RAM, ROM, EEPROM, flash memory, or any other medium that can be used to store desired program code in the form of instructions or data structures and that can be accessed by a computer).

Thus, instructions may be executed by one or more processors, such as one or more digital signal processors (DSPs), general purpose microprocessors, application specific integrated circuits (ASICs), field programmable logic arrays (FPGAs), and/or other equivalent integrated or discrete logic circuitry. Accordingly, the term “microprocessor” or “processor,” as used herein may refer to any of the foregoing structures or any other structure suitable for implementation of the techniques described herein. Also, the techniques could be fully implemented in one or more circuits or logic elements.

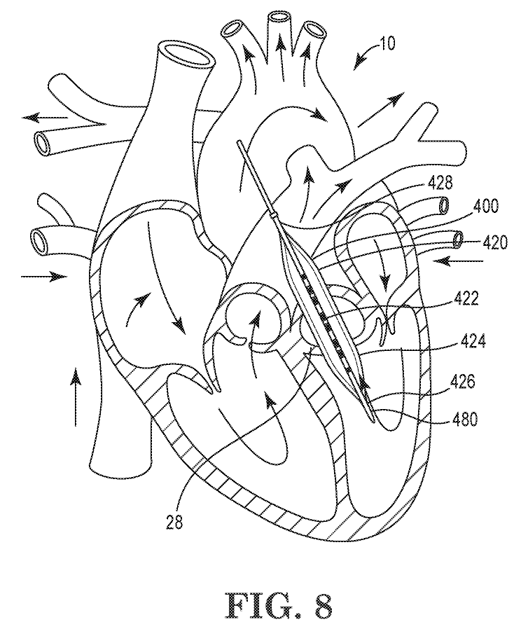

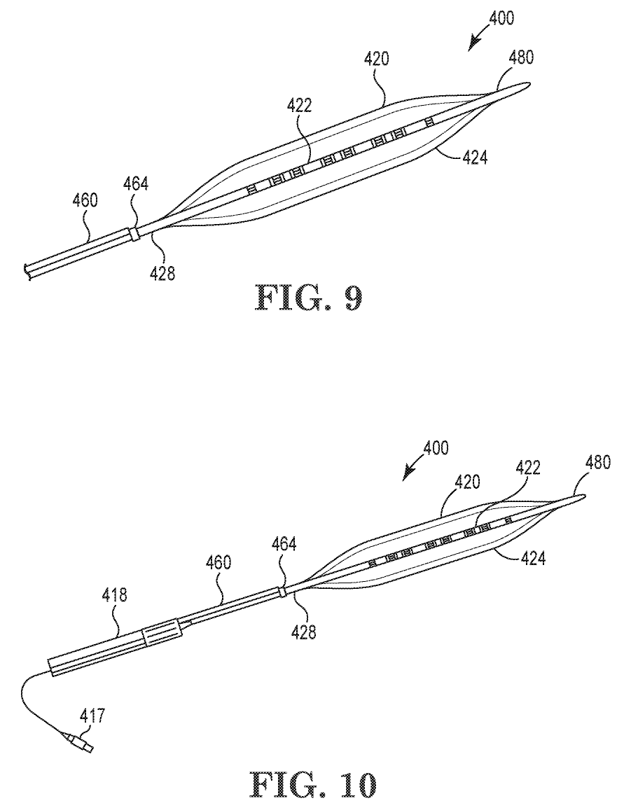

FIG. 8 illustrates another embodiment of a portion of a heart valve catheter system 400 in an appropriate position for treatment inside a heart 10, with FIGS. 9-14 illustrating a number of features and variations thereof. These embodiments generally include a catheter 420 with a deflectable distal tip 480 that can articulate such that a guide wire is not necessary to place the catheter in a desired location within a patient’s heart. However, it is contemplated that catheter 420 can utilize a guide wire during movement to a desired location (or part of the way to a desired location), after which the guide wire is removed. In either case, the deflectable distal tip 480 can be used either for large-scale movements or fine tuning of the system 400 within the patient.

FIG. 8 illustrates a general position of where catheter 420 can be placed in heart 10 in order to perform procedures on an aortic valve 28. The heart valve catheter system 400 can be similar in design to the system 100 of FIGS. 5 and 6, and/or the system 200 of FIG. 7. The heart valve catheter system 400 generally includes catheter 420, a central tube 426 with emitters 422, and a balloon 424. Although not illustrated relative to these embodiments, the system 400 may be connected to a power source, a controller, and a handle with a therapy delivery switch, for example, in accordance with other embodiments described herein.

As shown in FIGS. 8-12, each emitter 422 represents a pair of arcing electrodes, but could alternatively comprise optical or laser emitters. Optional marker bands may be provided to afford visualization and proper positioning by use of known imaging techniques. A catheter sheath 428 is connected at its distal end to a proximal end of the balloon 424. A proximal end of the sheath 428 is connected to a handle 418 from which a hub 417 extends (shown in FIG. 10 and FIG. 12, for example).

The central tube 426 can optionally include a guide wire lumen through which a guide wire can pass to assist in delivering the balloon 424 to a desired location along a guide wire. However, in accordance with the embodiments of FIGS. 8-14, the optional guide wire is not shown, which can be desirable to allow relatively free movement of the deflectable tip 480 and so that other features can be provided at a distal end of the catheter 420. Such features can include structure for anchoring of the catheter in a particular location, for example.

Sheath 428 surrounds the central tube 426 and defines a delivery lumen 429 through which saline can be controllably delivered for balloon inflation. The lumen 429 can provide a concentric space around the central tube 426 within which electrode wires can be run from the switch to the emitters 422. One or more push/pull wires 460 can also be provided in the lumen 429 to control the deflectable tip 480 at the distal end of catheter 420. The hub 417 can include any number of ports allowing the electrical wires 430 and/or push/pull wires 460 to pass into the lumen 429, along with saline for inflation and any number of other components, as desired.

With continued reference to the illustrated embodiments of FIGS. 8-14, one or more push/pull wires 460 are used in combination with at least one push/pull ring 464 to control movement of the deflectable tip 480. In particular, a distal end of each push/pull wire 460 is attached to a push/pull ring 464. A proximal end of each push/pull wire 460 is located proximal to the balloon 424 and connected to the handle 418. In other embodiments, controlling the movement of the push/pull rings is not via the handle but is provided by remote control in combination with a cartridge or similar (such as a base) that incorporates a push/pull wire movement mechanism.

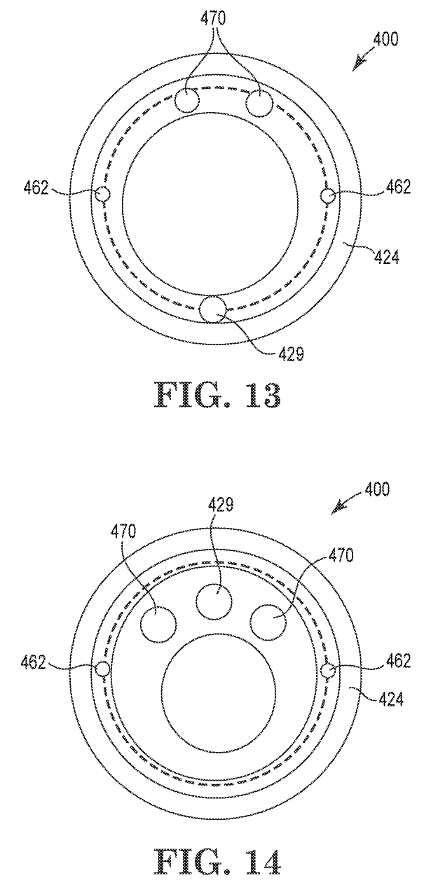

As is shown in the cross-sectional views of FIGS. 13-14, two lumens 462 are provided for push/pull wires 460. The lumens 462 are positioned 180 degrees, or approximately 180 degrees, from each other within the wall of the catheter 420. Alternatively, the push/pull wires 460 can instead be positioned so that they are not 180 degrees from each other. Additional lumens 470 are also illustrated, in which electrical wires described above can be positioned.

It is contemplated that only a single push/pull wire 460 (and therefore, only one corresponding lumen 462) can be used with a particular catheter, or that more than two of such wires 460 (and therefore, a corresponding number of lumens 462) may be used. If more than two push/pull wires 460 are used, they can be positioned equidistant from each other around the circumference of the catheter, or can be differently spaced around the catheter circumference. The lumens 462 extend along at least a portion of the length of the catheter 420, depending on the desired deflection of the deflectable tip 480.

The push/pull wires described herein can be made of a material (or combination of materials) that is sufficiently stiff that it can provide both the pulling and pushing functions described for moving a deflectable tip in a desired manner. In such cases, it is contemplated that the same wire can be used for both the pushing and pulling motions. In other exemplary configurations, separate wires may be provided for the “pushing” of the wire toward the distal end of the catheter and for the “pulling” of the wire toward the proximal end of the catheter such that more lumens are used for a particular catheter, and/or the lumens can house more than one wire. The push/pull wires may be made of a variety of materials that are relatively rigid and have a relatively high tensile strength, such as stainless steel (e.g., piano wire), for example. Additionally, the wire can be round, flat, or have a different cross-section. Although the term “push/pull wire” is used in this description, it is understood this may encompass a single strand or multiple strands (e.g., a cable).

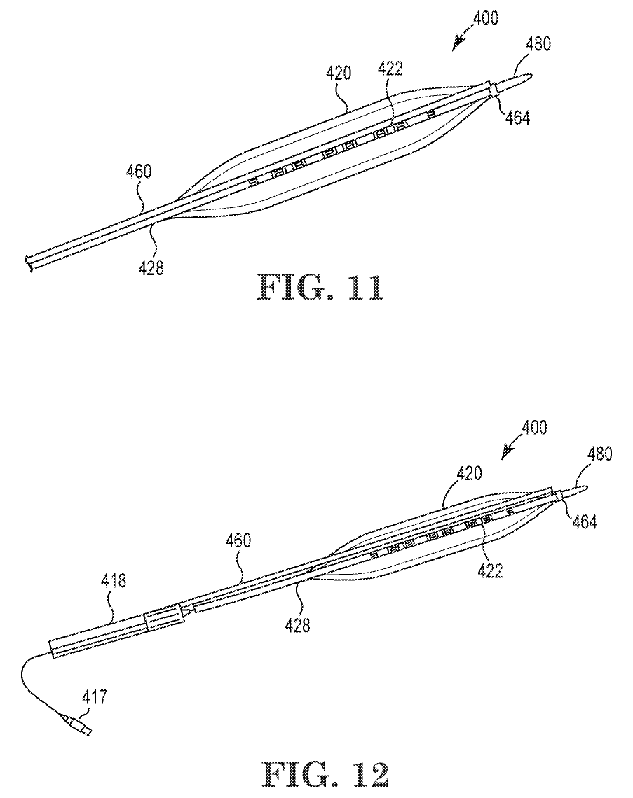

In FIGS. 9-10, push/pull ring 464 is illustrated proximal to the balloon 424 with a relatively short push/pull wire 460, while push/pull ring 464 is illustrated distal to the balloon 424 in FIGS. 11-12 with a relatively long push/pull wire 460. These different locations for the push/pull ring 464 will allow for different control of the distal deflectable tip 480 using similar techniques of moving the push/pull wires 460 relative to the length of the catheter 420, although the amount of movement required may be at least slightly different depending on the location of the ring 464.

Each push/pull ring 464 may include one of a wide variety of configurations and methods of attachment to the catheter 420. In one exemplary embodiment, each push/pull ring 464 is melted into the polymer of the catheter 420. In other exemplary embodiments, each push/pull ring 464 is attached to the catheter 420 via banding, gluing, or the like. It is further contemplated that each push/pull ring 464 can be inside or outside the catheter 420.

Each push/pull ring 464 may comprise a complete, closed circular shape, or can instead be provided as an open shape with a gap between its ends. In other embodiments, each push/pull ring 464 can be a shape that is at least slightly different from circular, such as oval, elliptical, or the like.

In order to control the deflectable tip 480, the proximal ends of the push/pull wires 460 are pushed or pulled to move the attached push/pull ring 464 toward or away from the deflectable tip 480. Moving the push/pull wires 460 in different directions and/or by different distances will curve the catheter in such a way that it deflects the tip 480 during placement of the catheter 420 in a desired position in the heart. Additional control of the deflectable tip can be achieved by varying the flexibility of the material proximal to the tip 480, such as by providing areas along the length with different durometers that are selected for a particular application, for example. In particular, portions with lower durometers can provide more flexibility along a particular section, while portion(s) with higher durometers will be less flexible or stiffer along that section. Such varying durometers will thereby be differently impacted by the movement of the push/pull wires and thereby allow for more specific deflection of the tip 480.

Braids, coils, and/or other materials can also be incorporated into the catheter wall, for example, to further change the flexibility of the device, and/or portions of the catheter can be treated to control their flexibility. It is further contemplated that areas of the catheter are weakened to control the flexibility of the device for positioning of the catheter 420 in a desired location. One exemplary manner of weakening includes providing slots (e.g., by laser cutting thereof) in a desired pattern along the catheter to influence the direction of catheter movement, including distal tip movement, when manipulating the push/pull wires.

In an embodiment, a portion of the area proximal to the balloon 424 is weakened and/or includes areas having different durometers to provide the desired flexibility discussed above. In a more specific embodiment, a portion of the area proximal to a push/pull ring that is proximal to a balloon is weakened and/or includes areas having different durometers to provide the desired flexibility to position the catheter in a desired location, as is discussed above.

In accordance with embodiments of the catheter 420, the deflectable tip 480 extends for a distance that corresponds to a desired amount of deflection and control for placement of the catheter 420 without the need for a guide wire. This distance can be at least partially determined by the location in the patient in which the catheter 420 will be positioned, the amount of control that is desired to reach the location, and/or to possibly reposition the tip once it is in its desired location.

As shown in FIGS. 8-14, the deflectable tip 480 includes a frustoconical shape at its distal end. It is contemplated that this end may instead have a different shape, including a sharper tip or a more rounded tip. The tip shape may be at least partially convex or concave, in other embodiments.

In accordance with embodiments described herein, the push/pull rings may include a radiopaque material that is detectible during and after positioning of the deflectable tip within a patient. Optional marker bands placed at a specific distance from any push/pull rings may be provided to afford further visualization and proper positioning by use of known imaging techniques.

A method for performing a medical procedure on a subject to modify (i.e. remove or reduce) calcifications on a heart valve of the subject can begin by positioning any of the high energy acoustic lithotripsy catheters that include a deflectable tip (as described herein) within a calcified heart valve. During this process, the deflectable tip is controlled via one or more push/pull wires attached to one or more push/pull rings that reconfigure the catheter to reach a desired location in the heart. In embodiments, movement of the push/pull wire(s) will deflect the distal portion of the catheter portion in a direction away from a longitudinal axis of the catheter assembly. Then, the balloon can be inflated and the operator can activate one or more or a series of pulses to break up calcium. Thereafter, the operator(s) can make a determination as to whether there is additional calcium on the heart valve to treat and the deflectable tip can again be reconfigured, if desired, via one or more push/pull wires attached to one or more push/pull rings that again reconfigure the catheter to reach another desired location in the heart.

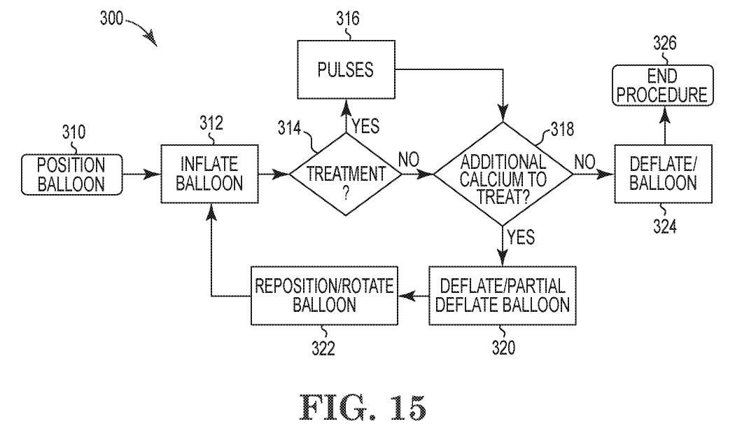

One such method is illustrated, for example, in the flow chart of FIG. 15, which shows a method 300 for performing a medical procedure on a subject to modify (i.e. remove or reduce) calcifications on a heart valve of the subject, for example using the heart valve catheter systems discussed herein. At step 310, the procedure begins by positioning the balloon catheter within a calcified heart valve, which can include manipulating push/pull ring(s) via push/pull wire(s) to deflect the tip of the balloon catheter. At step 312, the balloon is then inflated, with the surrounding calcium, disrupting the calcium. If the operator(s) then decides at step 314 to conduct high energy acoustic lithotripsy treatment, the operator can activate one or more, or a series of pulses at step 316 to break up the calcium. At step 318, the operator(s) determines whether there is additional calcium on the heart valve to treat. Such determination can include using fluoroscopy or any available imaging techniques, including echocardiography, CT, MRI, PET and SPECT, to view the heart valve and extent of calcification.

If additional locations of calcium require treatment, the balloon can then be deflated or partially deflated at step 320, which allows the operator to reposition and/or rotate the balloon at step 322. Such repositioning can include, for example, moving an axial position of the balloon in the heart and/or manipulating push/pull ring(s) via push/pull wire(s) to deflect the tip of the balloon catheter. The process can then be repeated until the operator determines at step 318 that the calcium has been fully or adequately treated. If no further treatment is needed at step 318, then at step 324 the balloon is deflated and the balloon can be removed, thus ending the procedure at step 326.

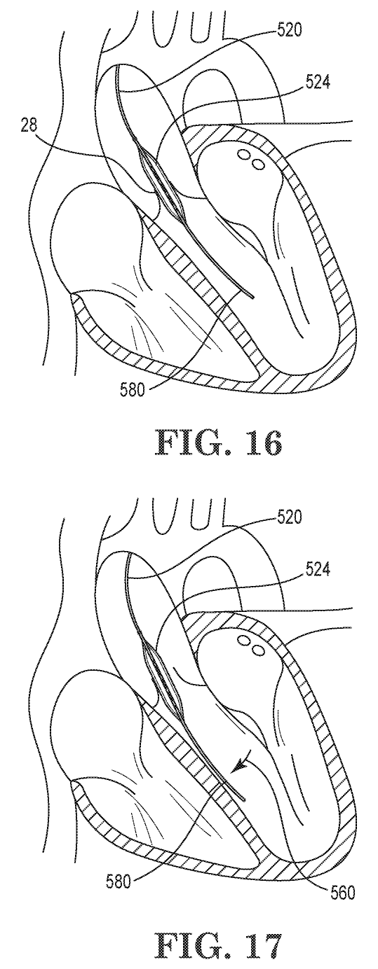

FIGS. 16-18 illustrate another embodiment of a portion of a heart valve catheter system 500 in an exemplary position for treatment inside a heart 10, with FIGS. 19-22 illustrating a number of features and variations thereof. The heart valve catheter system 500 can be similar in design to the system 100 of FIGS. 5 and 6, the system 200 of FIG. 7, and/or the system 400 of FIGS. 8 - 14. The heart valve catheter system 500 generally includes a catheter 520 with a deflectable distal tip 580 that can articulate such that a guide wire is not needed to place the catheter in a desired location within a patient’s heart. However, it is contemplated that catheter 520 can utilize a guide wire during movement to a desired location (or part of the way to a desired location), after which the guide wire is removed. In either case, the deflectable distal tip 580 can be used either for large-scale movements or fine tuning of the system 500 within the patient.

The catheter 520 can be steerable, such as is described herein relative to manipulating portions of the catheter to specific locations within the heart. With catheter 520, it is further contemplated that its balloon is designed to be “undersized” when in its inflated condition relative to the location to which it will be delivered. In an exemplary embodiment, the balloon can be inflatable to a diameter of 4-6 mm, although larger or smaller balloons are contemplated, depending on the size of the valve in which the balloon will be positioned. In an exemplary application of this configuration, the balloon can be positioned within an aortic valve that has a diameter of 20-30 mm, although the diameter of the valve may be significantly smaller due to calcification.

In practice, a size of a calcified heart valve is determined, and then an appropriately sized balloon is steered to a desired area within a subject to modify (i.e. remove or reduce) calcifications on a heart valve. The balloon is then inflated so that it does not fill the entire valve space in which it is positioned, such as less than 85 percent or less than 65 percent or less than 60 percent of the area in which it is positioned. Because the undersized balloon can provide relatively low pressure in the area of the leaflets of the valve in which it is positioned, the balloon can continue to be manipulated and repositioned by movement of a distal tip of the catheter even after it is in a generally desired area for treatment of calcification. In addition, when the balloon only partially fills the valve space, it is possible for blood to move past the balloon during the procedure, which is less disruptive to the patient.

The use of a balloon that is considered to be undersized relative to the valve space in which it is positioned can be provided with a balloon that, when fully inflated, is smaller than the space in which it is positioned. Alternatively, a balloon that can reach a much larger size can be used, wherein the size is carefully monitored during inflation so that it only reaches the desired size that is smaller than the space in which it is positioned.

FIG. 19 illustrates an exemplary heart valve catheter system 800 that is similar in design to the system 100 of FIGS. 5 and 6, the system 200 of FIG. 7, the system 400 of FIGS. 8-14, and/or the system 500 of FIGS. 16-18. The system 800 generally includes catheter 820, a central tube with emitters, and a steerable distal tip 880. The system can be provided without a balloon, or can further include a balloon at the distal tip 880 (i.e., on a deflectable catheter portion of the system), and/or can include a separate balloon that extends beyond the distal tip 880. The system 800 may be connected to a power source, a controller, and a handle 618 with a therapy delivery switch, for example, in accordance with other embodiments described herein.

The distal tip 880 is shown in a number of its possible configurations in FIG. 19, wherein movement to these configurations can be accomplished through the use of one or more push/pull wires in combination with at least one push/pull ring, for example. In such an embodiment, a distal end of each push/pull wire is attached to a push/pull ring, while a proximal end of each push/pull wire is connected to a handle 818. In other embodiments, controlling the movement of the push/pull rings is not via the handle but is provided by remote control in combination with a cartridge or similar (such as a base) that incorporates a push/pull wire movement mechanism. In other embodiments, movement of the distal tip is provided via other mechanisms or structures.

FIG. 16 illustrates one exemplary position of the catheter 520 placed in heart 10 in order to perform procedures on an aortic valve 28. As shown in this figure, the catheter 520 is positioned so that the balloon 524 is centered, or generally centered, within the aortic valve, with its distal tip 580 extending therefrom. Although the balloon 524 appears in the figure to fill, or generally fill, the aortic valve space, at least a small gap can be provided between the balloon 524 and the adjacent leaflets.

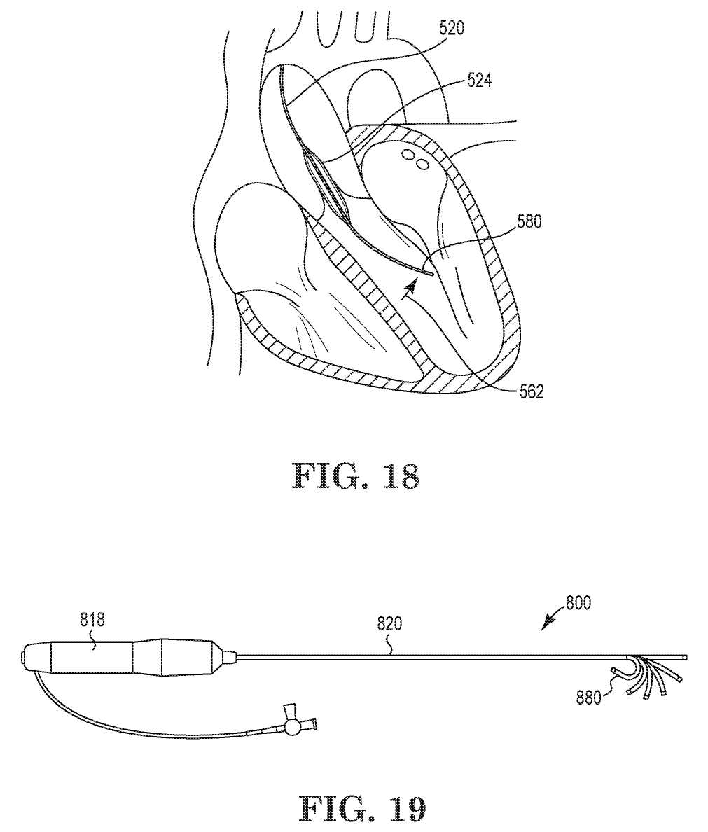

FIG. 17 illustrates catheter 520 with balloon 524 positioned to one side of an aortic valve opening. As shown by arrow 560, the distal tip 580 of the catheter 520 is controlled to move the balloon 524 to this left side of the valve. Similarly, FIG. 18 illustrates movement of the distal tip 580 toward the right side of the valve (see arrow 562), which will correspondingly move the balloon 524 toward the right side of the valve.

As with other embodiments discussed herein, the embodiment of FIGS. 16-22 uses high energy acoustic lithotripsy of a heart valve region. With this embodiment, one such device comprises a guide wire-compatible, fluid-filled balloon catheter that may include six lithotripsy emitters incorporated into the shaft of a balloon segment. A fluid filled balloon (e.g. a 50/50 saline contrast medium) is inflated to a desired size (which is “undersized” for the valve, as discussed above) and then electrical pulses are provided to the emitters that create high voltage sparks to provide the therapy against the lesion. Acoustic waves, also referred to herein as high energy acoustic waves, are created and the calcium is fractured.

With the smaller balloon size used in this embodiment, it is possible to treat an area within a valve, such as the area near one of the leaflets, then move the balloon to another area in that valve, such as the area near another of the leaflets, without deflating and reinflating the balloon. In this way, embodiments that use a smaller or undersized balloon can be manipulated for more targeted treatment of calcified areas of a valve. In addition to movement of the balloon to different areas of the valve circumference, such as by moving the balloon side-to-side or front-to-back, the catheter can be maneuvered to rotate the balloon within the valve space.

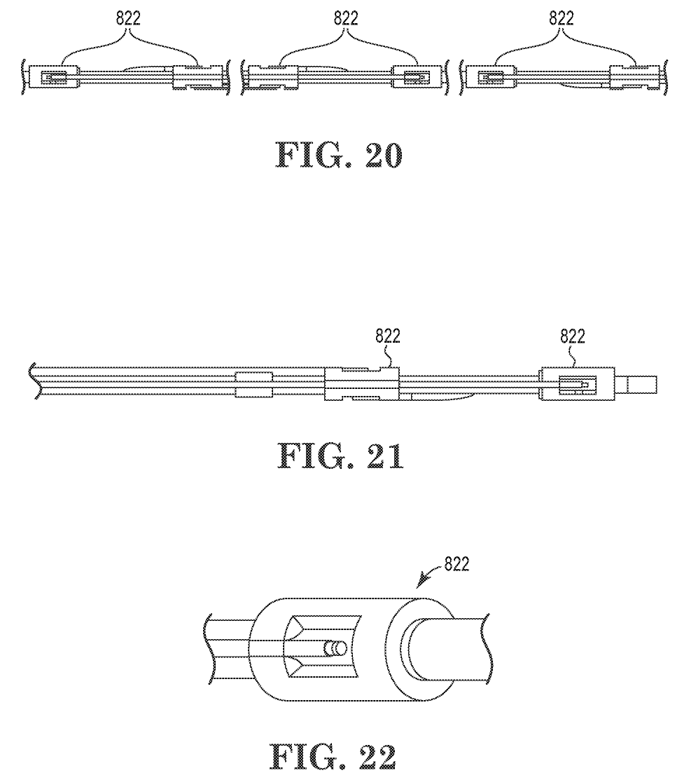

The embodiment illustrated in FIGS. 20-22 illustrates a portion of a catheter with six lithotripsy emitters 822 as they would be incorporated into the shaft of a balloon segment, although it is contemplated that more or less than six emitters are provided. It is noted that the descriptions of emitters 822 of this embodiment are also applicable to emitters of other embodiments described herein.

Each emitter 822 can include a pair of arcing electrodes, or could alternatively comprise optical or laser emitters. In an embodiment, each emitter 822 is rotated 180 degrees with respect to each adjacent emitter along the length of the catheter, although it is possible for adjacent emitters to be rotated a different amount relative to each other, or it is possible that adjacent emitters are not rotated relative to each other. The emitters 822 of this embodiment are arranged such that two emitters 822 are closely spaced from each other (e.g., 0.5mm – 5mm between emitters of a pair), with a substantially larger distance between adjacent pairs of emitters. In an embodiment, the emitter components may utilize a stainless-steel anode and/or cathode to reduce wear.

A method for performing a medical procedure on a subject to modify (i.e. remove or reduce) calcifications on a heart valve of the subject, in accordance with embodiments described herein (e.g., using the heart valve catheter systems discussed herein), can begin by positioning any of the high energy acoustic lithotripsy catheters that include a deflectable tip (as described herein) within a calcified heart valve. During this process, the deflectable tip is controlled to reconfigure the catheter to reach a desired location in the heart. In embodiments, movement of push/pull wire(s) will deflect the distal portion of the catheter portion in a direction away from a longitudinal axis of the catheter assembly. Then, the balloon can be inflated to be “undersized” relative to the valve space, and the operator can activate one or more or a series of pulses to break up calcium. Thereafter, the operator(s) can make a determination as to whether there is additional calcium on the heart valve to treat and the deflectable tip can again be reconfigured, if desired, so that the undersized balloon contacts a different location in the heart. The process then repeats until the operator determines that the calcium has been fully or adequately treated. If no further treatment is needed, then the balloon is deflated and the balloon can be removed, thus ending the procedure.

It is further contemplated that a procedure using an undersized balloon includes a catheter device (e.g., the device illustrated in FIG. 19) having only a single emitter at the end of the balloon. With such a device, the balloon can be positioned for treatment of a very specific area of a vessel.

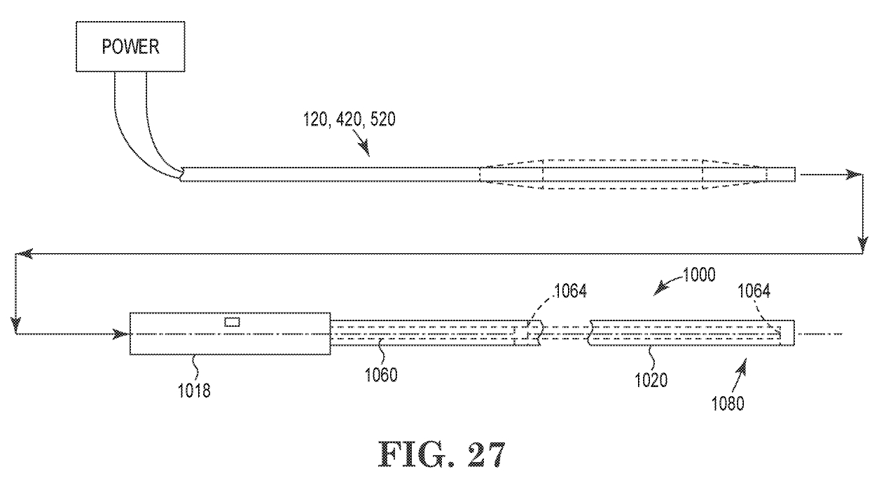

As discussed herein, the catheters can be steerable, such as is described relative to manipulating portions of the catheter to specific locations within the heart. The catheter (e.g., catheter 120, 420, 520) can be used with a steerable catheter 1000, as is schematically illustrated in FIG. 27. The steerable catheter 1000 can receive a catheter 120, 420, 520 within a lumen so that it can be advanced together with the steerable catheter 1000 through the patient’s anatomy and into the heart. The steerable catheter 1000 aids with placement of the catheter (e.g., catheter 120, 420, 520), which can either be non-steerable or deflectable or can optionally be steerable and/or include deflectable portions/tips. For example, once positioned in the heart, the steerable catheter 1000 can aid with positioning of the balloon, emitters, etc. to perform the therapeutic procedure and/or support the catheter 120, 420, 520 when such a catheter is steerable and/or deflectable.

The steerable catheter 1000 includes a catheter shaft 1020 and a handle 1018 operably coupled with the catheter shaft 1020 that can have a structure that is similar to that of catheter 420 exclusive of the balloon and emitters. The catheter shaft 1020 includes one or more push/pull wires 1060 that are provided to control a deflectable tip 1080 at the distal end of steerable catheter 1000, such as through the use of one or more push/pull rings 1064 similar to that provided for the operation of system 400. Alternatively, the push/pull wires 1060 can attach to one or more push/pull rings 1064 between the proximal and distal end of the steerable catheter 1000 to allow deflection or steering at a location other than the deflectable tip 1080.

In general, heart valve catheter systems described herein can include one or more emitters. In embodiments, at least one of the emitters is outside a balloon. For example, the catheter system can include an axial-firing emitter located distal to the balloon. The axial-firing emitter may be placed at various locations outside the balloon. The location may allow for the emitter to be spaced apart from tissue to avoid direct contact and excessive heating of tissue; however, the emitter could have various positions relative to the catheter tip. In an embodiment, an axial-firing emitter is at a distalmost location on the catheter. For example, the emitter may be located in the catheter tip at the distal end of the catheter shaft (e.g., an emitter can be recessed in the tip, a hollow cap may house the emitter, etc.).

Various non-limiting placements and structures of an axial-firing emitter include a number of exemplary embodiments, wherein pressure waves generated by an emitter may be used to help cross a tight lesion against which the catheter tip is placed. An emitter may be located along a taper of the tip, e.g., in the tip or mounted on an outer surface of the tapered tip. Integrating an emitter into the tip can shield the generated spark from the surrounding anatomy while producing waves pulses that can propagate forward to disrupt lesion calcifications and promote lesion crossing.

An axial-firing emitter can include one or more electrodes to generate an electrical spark. Alternatively, an axial-firing emitter can include an optical fiber to deliver an optical beam. For example, an optical fiber can deliver a laser to generate shockwaves. In either case, an emitter can generate acoustic waves in the exterior of the balloon. The acoustic waves can carry energy that originates in the exterior and remains in the exterior. For example, the energy can remain outside of, or substantially outside of, the balloon wall without any appreciable energy or acoustic waves traveling into the balloon. More particularly, the acoustic waves can travel in a distal axis direction, or a substantially distal axis direction, without any appreciable acoustic wave traveling from the interior of the balloon to the exterior of the balloon. The acoustic waves can propagate forward from a catheter tip. More particularly, an axial-firing emitter can be positioned to emit acoustic waves in an axial direction or substantially in an axial direction (e.g., along a longitudinal axis), without impinging on the balloon wall.

In an embodiment, a heart valve catheter system includes one or more emitters in a balloon. For example, a catheter system can include one or more radial-firing emitters mounted on an inner member of a catheter shaft in an interior of a balloon. As described above, radial-firing emitters can include an electrode to generate an electrical spark. For example, radial-firing emitters can include electrical spark gaps with wire pairs to generate the electrical spark. Alternatively, like the forward-firing electrode, the radial-firing electrodes may include optical focusing elements or optical targets with optical fibers. Accordingly, radial-firing emitters can be activated to emit acoustic waves in a radial direction or substantially in a radial direction (e.g., perpendicular to the axial direction of the axially fired acoustic waves) when the balloon is inflated.

Each emitter (e.g., an axial-firing emitter or a radial-firing emitter) can include electrode pairs to generate an electrical spark that induces a pressure wave. For example, an axial-firing emitter can include a first electrode pair, and one or more radial-firing emitters can include a second electrode pair and/or additional electrode pair(s)). Embodiments of electrode pairs are disclosed in U.S. Patent Application No. 63/735,842, filed December 18, 2024, and entitled “Intravascular Lithotripsy Catheter Having Directional Energy Delivery,” and PCT Publication No. WO 2024/081361 A1, internationally filed October 12, 2023, and entitled “Intravascular Lithotripsy Devices and Systems with Forward Facing Electrodes and Flex Circuit Arrangements,” which applications are incorporated herein by reference in their entireties.

Electrode pairs can be controlled by an electrode controller to supply electrical (or other) energy pulses. An electrode controller can be implemented apart from an enclosure of a catheter control system, such as in the control handle or a console of the control system accessible by a health care provider. As described herein, the electrode controller can independently control each of the electrode pairs of the system. For example, a first electrode pair could be actuated without actuating other electrode pairs. Actuating an electrode pair comprises delivering an energy pulse to the electrode pair causing an electrical arc and concomitant emission of a pressure wave. Accordingly, actuation of a first electrode pair can emit a pressure wave in the axial direction without emitting pressure waves from second and third electrode pairs in the radial direction, for example.

As described herein, emitters of a heart valve catheter system can generate sparks or manipulate optical beams. The delivered energy can produce shockwaves that propagate outward (e.g., forward or laterally) toward a target lesion. It will be appreciated that such pressure pulses may be generated by alternative mechanisms. For example, pressurized gas may be delivered in bursts internal or external to a balloon to generate short pressure pulses. The pressure pulses may also be focused (e.g., through refractive or reflective elements) or unfocused. In an embodiment, acoustic waves emitted in an axial direction are focused to target a narrow profile in the forward direction, and acoustic waves emitted in a radial direction are unfocused to target a broader circumferential arc or profile in the lateral direction.

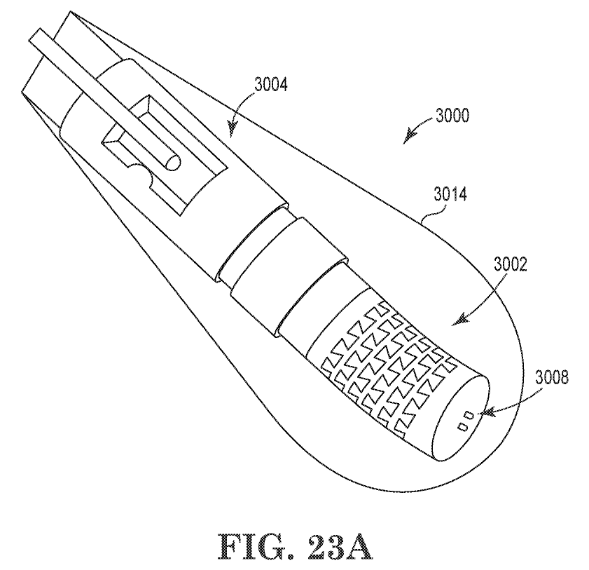

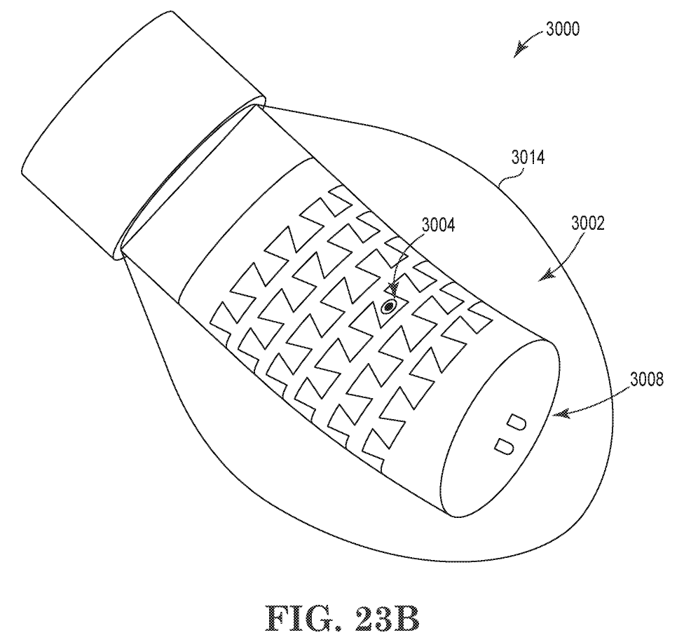

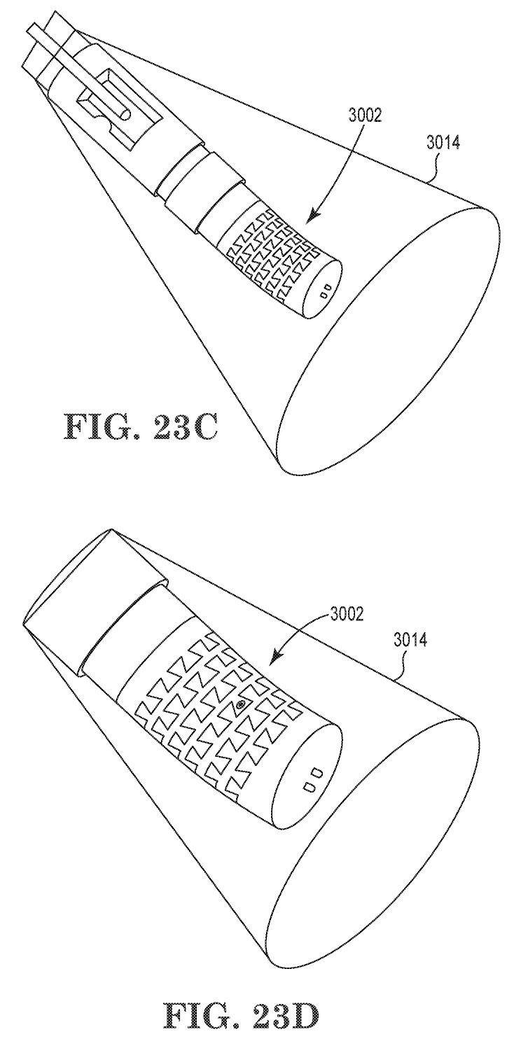

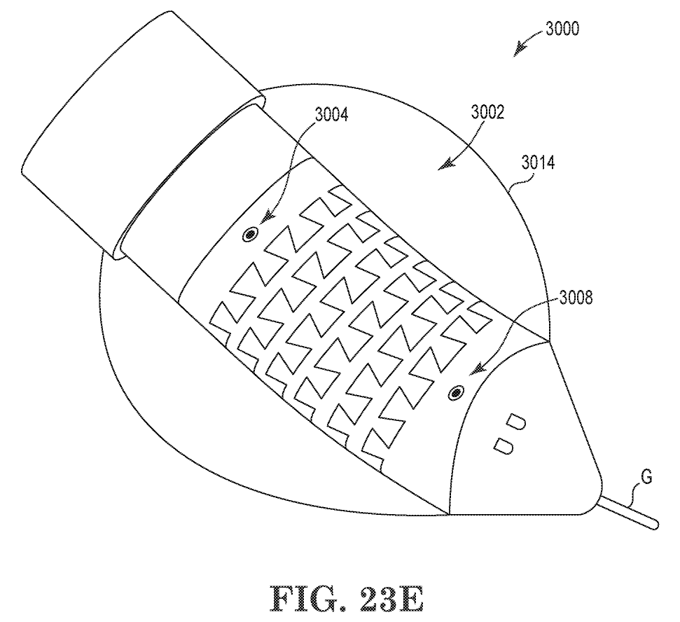

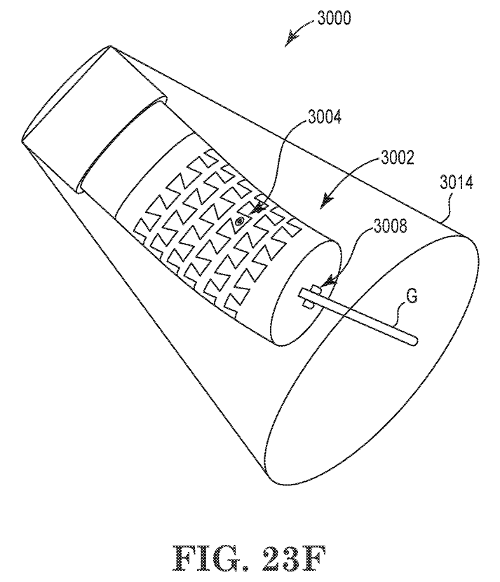

Referring now to FIGS. 23A-23D, additional examples of a catheter tip including both one or more axial-firing emitters and one or more radial or distally firing emitters are illustrated. In this example, a heart valve catheter system 3000 is shown. The catheter system 3000 is disposed at a distal end of a catheter shaft and includes a heart valve catheter system, one or more lithotripsy emitters 3004 and 3008, and a balloon 3014. In closed-balloon embodiments (see FIG. 23A), the balloon 3014 surrounds the lithotripsy emitters 3004, 3008 and is configured to retain a dielectric or conductive fluid during operation. In open-balloon embodiments (see FIGS. 23C-23D), the balloon 3014 defines an open flow path that permits continuous fluid exchange across the emitters.

A flex tip 3002 is configured to flex or steer selectively in one or more directions. In some embodiments, the flex tip 3002 includes one or more slots, notches, or grooves (collectively “slots”) formed therein, such as by laser cutting a solid tubular member, to form a flexible segment.

In some embodiments, four steering wires (although other numbers of wires may be used) are disposed circumferentially around the elongate flexible member. Each wire is anchored near or distal to the slotted region. Selective tensioning or compression of individual wires from a proximal handle produces bidirectional or omnidirectional deflection of the flex tip 3002. In alternative embodiments, a proximal control handle includes a rotatable deflection collar coupled to an internal deflection element extending along the shaft such that rotation of the collar about its axis translates the control element axially, inducing curvature of the distal segment. Counter-rotation returns the segment toward neutral. A locking assembly may be provided to maintain a selected curvature during catheter manipulation or device exchange. Suitable steering mechanisms may be of the type used in commercially available steerable introducers or other equivalent deflection systems.

Steering or bending of the flex tip 3002 repositions the lithotripsy emitters 3004 and/or 3008 toward a target region such as a calcified lesion. By positioning an emitter within several millimeters of the target, the resulting acoustic shockwaves can deliver increased localized pressure (e.g., increases of 10–50 percent at 1–5 mm standoff), enhancing disruption of calcific deposits.

In the embodiment of FIG. 23A, two different types of emitters are shown. A distal emitter 3008 is implemented as an axial-firing spark-gap disposed at the distal face of the flex tip 3002. Conductors extend from the heart valve catheter system through the catheter shaft to the distal emitter. The distal emitter 3008 may include opposed or coaxially aligned electrode tips forming a spark gap of 0.05–0.5 mm, or approximately 0.05–0.5 mm, energized at 0.5–5 kV, to produce an axial acoustic shockwave.

Distal electrode materials may include one or more of platinum, palladium, tungsten, gold, and/or stainless steel to provide durability for extended discharge cycling. The electrodes may be insulated by dielectric sleeves of polyimide or PEEK, and the balloon fluid can serve as a thermal sink to dissipate heat. Such materials and configurations are particularly advantageous when the distal emitter 3008 is used for a large number of discharge cycles during treatment.

In other embodiments, the distal emitter 3008 may be designed for limited-use applications, for example when a small number of chronic total occlusions (CTOs) are encountered. In such cases, lower-cost or less durable electrode materials may be employed without significant performance loss. While the distal emitter 3008 is depicted as a spark-gap formed by wire ends, other emitter geometries (e.g., ring-tip, coaxial, or recessed-tip configurations) may be employed to achieve a desired shockwave pattern.

Also shown in FIG. 23A, an emitter pair 3004 provides radial emission. Each pair may be implemented in the manner of other emitter pairs described herein. Although one emitter is visible in the figure, a second, diametrically opposed emitter is provided on the opposite side of the balloon. The number and arrangement of emitters can vary; in some embodiments a single radial emitter or multiple circumferentially spaced emitters are provided.

The distal emitter 3008 and the emitter pair 3004 are independently controllable, allowing selective operation of axial-firing and radial-firing modes. Each emitter or emitter group may be connected to a dedicated high-voltage capacitor, charging circuit, and switching device. A control module may be configured to activate the emitters individually, simultaneously, or in interleaved sequences according to a selected treatment mode.

An alternative configuration is illustrated in FIG. 23B, in which all or a portion of the flex tip 3002 itself serves as an electrode of the emitter pair 3004. A first hole is formed through the flex-tip wall, and a first conductor disposed in the hole forms a first electrode. A conductive portion of the flex-tip wall acts as a counter-electrode. A second hole and conductor may define a second emitter spaced axially or circumferentially from the first. Conductors extend through an internal lumen or along an inner member to the proximal heart valve catheter system.

While the embodiments of FIG. 23A and FIG. 23B illustrate closed-balloon configurations, open-balloon embodiments are shown in FIG. 23C and FIG. 23D. In these embodiments, the balloon 3014 may include one or more fluid inlet and outlet openings that allow continuous supply and evacuation of liquid during operation to remove gas bubbles generated by emitter discharges. The balloon or flow shroud may be secured to the catheter assembly by adhesive bonding or thermal welding at proximal and distal attachment regions.

FIG. 23E and FIG. 23F illustrate embodiments of a heart valve catheter system 3000 incorporating a guidewire G extending through a flex tip 3002 that supports both radial and axial lithotripsy emitters. The guidewire G provides a central lumen along which the catheter may be advanced to a target lesion within a vessel. The distal region of the catheter includes the flex tip 3002, which is configured to bend or steer to facilitate accurate positioning of the emitters relative to a calcified lesion. A radial emitter pair 3004 is disposed along or within the wall of the flex tip to emit acoustic energy radially toward surrounding calcified tissue. A distal axial-firing emitter 3008 is located near the distal end of the tip to generate forward-directed acoustic shockwaves along the guidewire path. As shown in FIG. 23E, the balloon 3014 may be a closed-balloon configuration configured to contain a dielectric or conductive fluid that supports spark generation and acoustic wave propagation. In this embodiment, while a single emitter is visible, the distal emitter 3008 may be implemented as part of an emitter pair with a corresponding emitter on an opposing side of the flex tip 3002. Note that electrical isolation may be needed between certain segments of the flex tip 3002.

In contrast, FIG. 23F illustrates an open-balloon embodiment, in which the balloon 3014 defines an open flow path that allows continuous exchange of fluid across the emitters during operation to remove gas bubbles produced by discharge events and to maintain consistent acoustic coupling. In both embodiments, the guidewire lumen provides trackability and alignment during advancement, while the combined radial and axial emitters permit both forward and circumferential lithotripsy energy delivery to efficiently cross and remodel calcified occlusions.

While certain examples are shown here, it should be appreciated that other emitter configurations, such as those previously disclosed, could be used with the guidewire configurations illustrated.

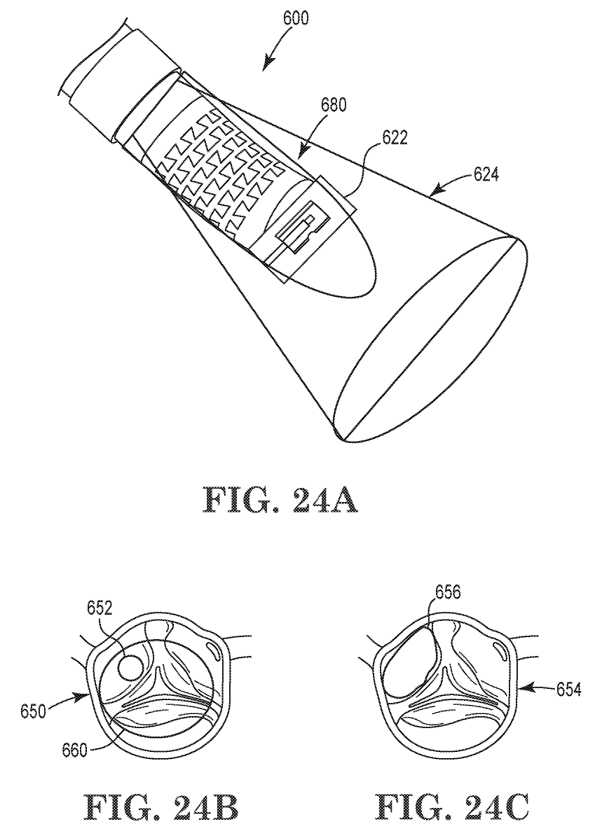

FIGS. 24A-24C illustrate multiple features and embodiments of a distal tip portion of a heart valve catheter system, and applications thereof. FIG. 24A illustrates an emitter 622 at a deflectable distal tip portion 680 of a heart valve catheter system 600 that is similar to heart catheter systems 100, 200, 400, 500, wherein the tip portion can articulate such that a guide wire is not necessary to place the catheter in a desired location within a patient’s heart. However, it is contemplated that the catheter can utilize a guidewire during movement to a desired location (or part of the way to a desired location), after which the guide wire is removed. In either case, the deflectable distal tip 680 can be used either for large-scale movements or fine tuning of the system 600 within the patient.

The distal tip portion 680 includes emitter 622 at or near its end. The emitter 622 can be attached to a flex tip in a variety of ways, such as laser welding. The system can include a relatively focal balloon 624 that is attached to the shaft, such as by gluing it to the shaft. The balloon can be cusp-shaped or oval-shaped, for example, to accommodate placement in the desired valve area of the heart. When used with other valves, the shape of the balloon can be changed to accommodate those particular valve opening and leaflet shapes.

FIGS. 24B-24C illustrate top views of exemplary stenotic aortic valves 650 and 654, respectively, wherein such aortic valves could normally have a diameter of 20mm, or approximately 20mm, as shown with the larger solid circle 660 of FIG. 24B. An exemplary embodiment of emitter 622 can be 5mm long, or approximately 5mm long, such that it is positionable in the area of the smaller solid circle 652 of FIG. 24B, which is 4mm in diameter, or approximately 4mm in diameter. With a balloon that is oval-shaped or cusp-shaped, it can be inflated to match the shape 656 shown in FIG. 24C.

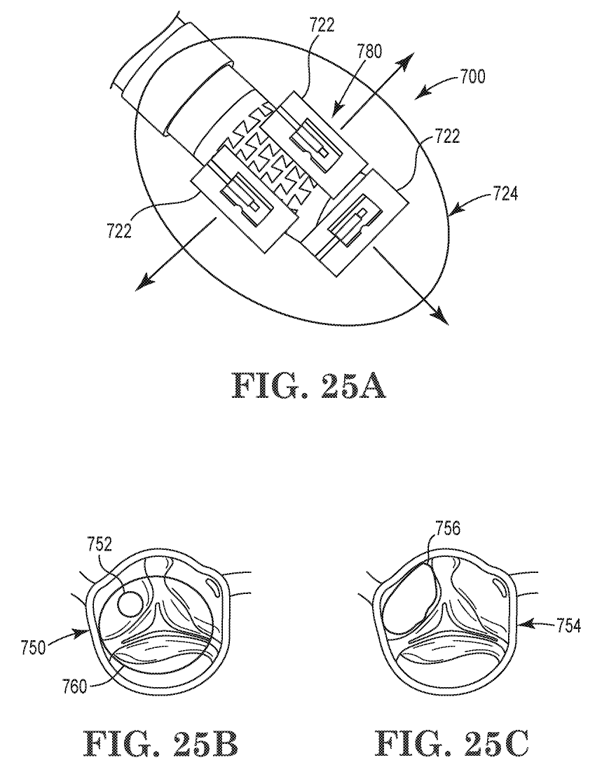

FIGS. 25A-25C illustrate multiple features and embodiments of a distal tip portion of a heart valve catheter system 600 that is similar to heart catheter systems 100, 200, 400, 500, and applications thereof. FIG. 25A illustrates an emitter 722 at an end and additional emitters 722 on the sides of the flex tip at a deflectable distal tip portion 780 of a heart valve catheter system 700. The tip portion can articulate such that a guide wire is not necessary to place the catheter in a desired location within a patient’s heart. However, it is contemplated that the catheter can utilize a guide wire during movement to a desired location (or part of the way to a desired location), after which the guide wire is removed. In either case, the deflectable distal tip 780 can be used either for large-scale movements or fine tuning of the system 700 within the patient.

The distal tip portion 780 includes emitters 722 at or near its end and along its sides. The emitters 722 can be attached to a flex tip in a variety of ways, such as laser welding. The system can include a relatively focal balloon 724 that is attached to the shaft, such as by gluing it to the shaft. The balloon can be cusp-shaped or oval-shaped, for example, to accommodate placement in the desired valve area of the heart. When used with other valves, the shape of the balloon can be changed to accommodate those particular valve opening and leaflet shapes.

FIGS. 25B-25C illustrate top views of exemplary stenotic aortic valves 750 and 754, respectively, wherein such an aortic valve could normally have a diameter of 20mm, or approximately 20mm, as shown with the larger solid circle 760 of FIG. 25B. An exemplary embodiment of emitters 722 can be 5mm long, or approximately 5mm long, such that the tip is positionable in the area of the smaller solid circle 752 of FIG. 25B, which is 4mm in diameter, or approximately 4mm in diameter. With a balloon that is oval-shaped or cusp-shaped, it can be inflated to match the shape 756 shown in FIG. 25C, thereby being able to treat calcium deposits at the bottom and/or up the sides of the valve leaflet, base, and/or wall.

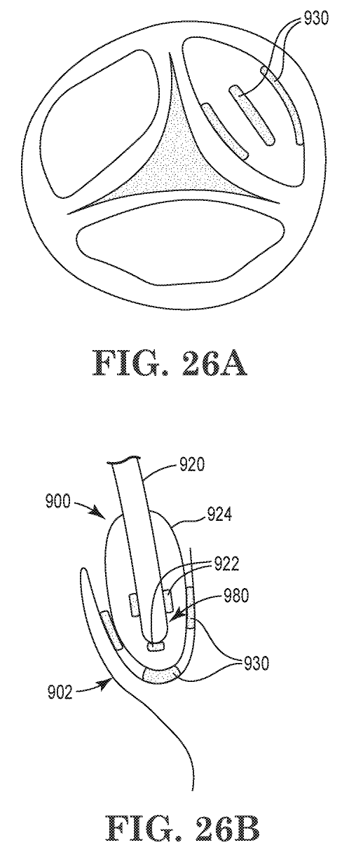

FIG. 26A illustrates a partially open aortic heart valve with multiple calcium deposits 930 in one portion of the heart. FIG. 26B illustrates treatment of the calcium deposits 930 relative to a leaflet 902 with a heart valve catheter system 900 that is similar to heart catheter systems 100, 200, 400, 500. System 900 includes a catheter 920 with emitters 922 at a distal tip portion and along its sides. System 900 includes a deflectable distal tip 980 that can articulate such that a guide wire is not necessary to place the catheter in a desired location within a patient’s heart. However, it is contemplated that catheter can utilize a guide wire during movement to a desired location (or part of the way to a desired location), after which the guide wire is removed. In either case, the deflectable distal tip 980 can be used either for large-scale movements or fine tuning of the system 900 within the patient. This system 900 includes emitters at the end and sides of the flex tip, similar to that described above relative to the system of FIG. 25A. The system includes a balloon 824 that is attached to the shaft.

Other aspects of high energy acoustic lithotripsy systems of the present invention and other related features thereof are described in U.S. provisional applications titled: “HIGH ACOUSTIC ENERGY CATHETER SYSTEM FOR HEART VALVE CALCIUM MODIFICATION”, Application Serial No. 63/754, 811, filed February 6, 2025; “SYSTEMS AND METHODS FOR CONTROLLED DELIVERY OF ENERGY BY A CATHETER SYSTEM BASED ON A HEARTBEAT CYCLE”, Application Serial No. 63/754,798, filed February 6, 2025; and “SYSTEMS AND METHODS FOR CONTROLLED DELIVERY OF ENERGY BY A CATHETER SYSTEM BASED ON A HEARTBEAT CYCLE”, Application Serial No. 63/856,958, filed August 4, 2025. Each of these provisional applications are fully incorporated herein in their entirety by reference.

The description of the invention and its applications as set forth herein is illustrative and is not intended to limit the scope of the invention. Features of various embodiments may be combined with other embodiments within the contemplation of this invention. Variations and modifications of the embodiments disclosed herein are possible, and practical alternatives to and equivalents of the various elements of the embodiments would be understood to those of ordinary skill in the art upon study of this patent document. These and other variations and modifications of the embodiments disclosed herein may be made without departing from the scope and spirit of the invention.

The following clauses present further examples, wherein such clauses are presented only by way of example and are not intended to limit the scope of the invention in any way. Further, any example configuration can be combined with one or more of the example configurations:

Clause 1. An elongate catheter for providing high energy acoustic waves to a heart valve region of a subject, the catheter system comprising:

an inflatable balloon at a distal portion of the catheter system, the inflatable balloon adapted to:

traverse a vessel of the subject in a radial retracted configuration to reach a treatment site in the heart valve region; and

receive fluid to inflate to an expanded configuration at the treatment site;

a deflectable distal tip portion distal to the inflatable balloon;

an emitter within the inflatable balloon;

a conductive pathway for supplying energy to the emitter to produce a spark to generate high energy acoustic waves within fluid inflating the balloon; and

a movable control member operatively connected to the distal portion for deflecting the distal tip portion in a direction away from the longitudinal axis of the catheter system.

Clause 2. The catheter of Clause 1, wherein the movable control member comprises at least one push/pull ring positioned along a length of the catheter system and proximal to the distal tip portion.

Clause 3. The catheter of Clause 2, wherein the movable control member further comprises at least one push/pull wire attached to and extending in a proximal direction from the at least one push/pull ring, wherein movement of the at least one push/pull ring is controlled by movement of the at least one push/pull wire to deflect the distal tip portion.

Clause 4. The catheter of Clause 2 or Clause 3, wherein the at least one push/pull ring is positioned distal to the inflatable balloon.

Clause 5. The catheter of Clause 2 or Clause 3, wherein the at least one push/pull ring is positioned proximal to the inflatable balloon.

Clause 6. The catheter of any of Clauses 3 - 5, wherein the at least one push/pull wire is moveable in an axial direction relative to a length of the catheter system.

Clause 7. The catheter of any of Clauses 3 – 6, wherein the at least one push/pull wire comprises two push/pull wires.

Clause 8. The catheter of Clause 7, wherein the two push/pull wires are located 180 degrees from each other relative to a circumference of the catheter system.

Clause 9. The catheter of Clause 1, wherein the emitter comprises at least two electrodes with a gap therebetween, the electrodes being spaced axially along a longitudinal axis of the catheter.

Clause 10. A method of treatment and repair of a calcified heart valve of a patient with a heart valve catheter system having a steerable distal tip portion, the method comprising:

inserting an inflatable balloon of the catheter system into a patient’s heart and moving the inflatable balloon to the calcified heart valve, the catheter system comprising a steerable distal tip portion that extends distally beyond a distal end of the inflatable balloon, the catheter system further including first and second electrodes located inside the balloon to form a first emitter, the first and second electrodes connected at the proximal portion of the catheter to a high voltage pulse generator to generate a high voltage pulse;

manipulating the distal tip portion to steer the catheter system to a first treatment location within the calcified heart valve;

inflating the balloon such that an exterior surface of the balloon is in contact with one or more leaflets of the calcified heart valve; and

providing a high voltage pulse from a high voltage pulse generator to create a spark from the first emitter for creating an energy wave for propagation through the fluid within the balloon to provide force transmitted through the balloon to break up the calcifications at the first treatment location.

Clause 11. The method of Clause 10, wherein the step of manipulating the deflectable tip portion to the treatment location within the calcified heart valve comprises moving at least one push/pull wire of the catheter system, wherein the at least one push/pull wire extends proximally from at least one push/pull ring.

Clause 12. The method of Clause 10 or Clause 11, wherein inserting the inflatable balloon into the heart includes inserting the inflatable balloon in a deflated state and inflating the balloon once the balloon is positioned at the calcified heart valve.

Clause 13. The method of any of Clauses 10 - 12, further comprising a step of controlling the high voltage pulse generator to control an amount and frequency of the high voltage pulse.

Clause 14. The method of any of Clauses 10-13, further comprising the steps of:

providing a plurality of high voltage pulses from the high voltage pulse generator to create a plurality of sparks from the first emitter for creating a plurality of energy waves for propagation through the fluid within the balloon to provide force to break up the calcifications at the first treatment location; and

controlling the high voltage pulse generator to control an amount and frequency of the plurality of high voltage pulses.

Clause 15. The method of any of Clauses 10 - 14, further comprising a step of manipulating the distal tip portion to steer the catheter system to a second treatment location within the calcified heart valve.

Clause 16. The method of any of Clauses 10 - 15, further comprising the steps of:

at least partially deflating the balloon;

moving the catheter system within the patient’s heart;

manipulating the distal tip portion to steer the catheter system to a second treatment location within the calcified heart valve.

Clause 17. A method of treatment and repair of a calcified heart valve of a patient with a heart valve catheter system having a steerable distal tip portion, the method comprising:

inserting an inflatable balloon of the catheter system into a patient’s heart and moving the inflatable balloon to the calcified heart valve, the catheter system comprising a steerable distal tip portion that extends distally beyond a distal end of the inflatable balloon, the catheter system further including at least two electrodes located inside the balloon to form a first emitter, the at least two electrodes connected at the proximal portion of the catheter system to a high voltage pulse generator to generate a high voltage pulse;

inflating the balloon to less than a size of an inner area of the calcified heart valve;

deflecting the distal tip portion to a first treatment location so that a portion of an exterior surface of the balloon is in contact with one or more leaflets of the heart valve; and

providing a high voltage pulse from the high voltage pulse generator to create a spark from the first emitter for creating an energy wave for propagation through the fluid within the balloon to provide force transmitted through the balloon to break up any calcification at the first treatment location.

Clause 18. The method of Clause 17, further comprising steps of manipulating the heart valve catheter system to a second treatment location by steering the distal tip portion inside the heart without deflating the balloon, and then providing a high voltage pulse from a high voltage pulse generator to create a spark from an emitter for creating an energy wave for propagation through the fluid within the balloon to provide force from the balloon to break up the calcifications at the second treatment location.

Clause 19. The method of Clause 17 or Clause 18, wherein the step of inserting the inflatable balloon into the heart includes inserting the inflatable balloon in a deflated state and inflating the balloon once the balloon is positioned in the interior of the heart.

Clause 20. The method of claim of any of Clauses 17 - 19, wherein the step of inflating the balloon to less than the size of the inner area of the heart valve comprises inflating the balloon to less than 80 percent of the size of the inner area of the heart valve.

Clause 21. The method of any of Clauses 17 - 20, wherein the at least two electrodes comprise six electrodes that form the first emitter, a second emitter, and a third emitter, and further comprising providing a high voltage pulse from the high voltage pulse generator to create a spark from the second and third emitters for creating at least one other energy wave for propagation through the fluid within the balloon to provide force to break up the calcification at the first treatment location.

Clause 22. The method of Clause 21, wherein the six electrodes are arranged in pairs that are spaced from each other by a distance that is smaller than the distance between adjacent pairs of electrodes.

Clause 23. An elongate catheter system for providing high energy acoustic waves to a heart valve region of a subject, the catheter system comprising:

an inflatable balloon at a distal portion of the catheter system, the inflatable balloon adapted to:

traverse a vessel of the subject in a radial retracted configuration to reach a treatment site in the heart valve region; and

receive fluid to inflate to an expanded configuration at the treatment site;

a deflectable distal tip portion distal to the inflatable balloon;

an emitter within the inflatable balloon;

a conductive pathway for supplying energy to the emitter to generate high energy acoustic waves within fluid inflating the balloon;

a movable control member operatively connected to the distal portion for deflecting the distal tip portion in a direction away from the longitudinal axis of the catheter system;

a pulse generator for creating a spark from the emitter to create an energy wave for propagation through fluid within the balloon and provide force to break up calcifications; and

a control module for controlling the pulse generator.

Clause 24. The catheter system of any of Clauses 1 – 8, wherein the at least two electrodes are connected to a high voltage pulse generator at a proximal portion of the catheter system, the high voltage pulse generator configured to generate a high voltage pulse.

Clause 25. The catheter system of Clauses 1 – 8 or Clause 24, wherein the at least two electrodes comprise six electrodes.

Clause 26. The catheter system of Clause 25, wherein the six electrodes are arranged in pairs that are spaced from each other by a distance that is smaller than the distance between adjacent pairs of electrodes.

1. An elongate catheter for providing high energy acoustic waves to a heart valve region of a subject, the catheter system comprising:
an inflatable balloon at a distal portion of the catheter system, the inflatable balloon adapted to:
traverse a vessel of the subject in a radial retracted configuration to reach a treatment site in the heart valve region; and
receive fluid to inflate to an expanded configuration at the treatment site;
a deflectable distal tip portion distal to the inflatable balloon;
an emitter within the inflatable balloon;
a conductive pathway for supplying energy to the emitter to produce a spark to generate high energy acoustic waves within fluid inflating the balloon; and
a movable control member operatively connected to the distal portion for deflecting the distal tip portion in a direction away from the longitudinal axis of the catheter system.

2. The catheter of claim 1, wherein the movable control member comprises at least one push/pull ring positioned along a length of the catheter system and proximal to the distal tip portion.

3. The catheter of claim 2, wherein the movable control member further comprises at least one push/pull wire attached to and extending in a proximal direction from the at least one push/pull ring, wherein movement of the at least one push/pull ring is controlled by movement of the at least one push/pull wire to deflect the distal tip portion.

4. The catheter of claim 2, wherein the at least one push/pull ring is positioned distal to the inflatable balloon.

5. The catheter of claim 2, wherein the at least one push/pull ring is positioned proximal to the inflatable balloon.

6. The catheter of claim 3, wherein the at least one push/pull wire is moveable in an axial direction relative to a length of the catheter system.

7. The catheter of claim 3 wherein the at least one push/pull wire comprises two push/pull wires.

8. The catheter of claim 7, wherein the two push/pull wires are located 180 degrees from each other relative to a circumference of the catheter system.

9. The catheter of claim 1, wherein the emitter comprises at least two electrodes with a gap therebetween, the electrodes being spaced axially along a longitudinal axis of the catheter.

10. A method of treatment and repair of a calcified heart valve of a patient with a heart valve catheter system having a steerable distal tip portion, the method comprising:
inserting an inflatable balloon of the catheter system into a patient’s heart and moving the inflatable balloon to the calcified heart valve, the catheter system comprising a steerable distal tip portion that extends distally beyond a distal end of the inflatable balloon, the catheter system further including first and second electrodes located inside the balloon to form a first emitter, the first and second electrodes connected at the proximal portion of the catheter system to a high voltage pulse generator to generate a high voltage pulse;
manipulating the distal tip portion to steer the catheter system to a first treatment location within the calcified heart valve;
inflating the balloon such that an exterior surface of the balloon is in contact with one or more leaflets of the calcified heart valve; and
providing a high voltage pulse from the high voltage pulse generator to create a spark from the first emitter for creating an energy wave for propagation through the fluid within the balloon to provide force transmitted through the balloon to break up the calcifications at the first treatment location.

11. The method of claim 10, wherein the step of manipulating the steerable distal tip portion to the treatment location within the calcified heart valve comprises moving at least one push/pull wire of the catheter system, wherein the at least one push/pull wire extends proximally from at least one push/pull ring.

12. The method of claim 10, wherein inserting the inflatable balloon into the heart includes inserting the inflatable balloon in a deflated state and inflating the balloon once the balloon is positioned at the calcified heart valve.

13. The method of claim 10, further comprising a step of controlling the high voltage pulse generator to control an amount and frequency of the high voltage pulse.

14. The method of claim 10, further comprising the steps of:
providing a plurality of high voltage pulses from the high voltage pulse generator to create a plurality of sparks from the first emitter for creating a plurality of energy waves for propagation through the fluid within the balloon to provide force to break up the calcifications at the first treatment location; and
controlling the high voltage pulse generator to control an amount and frequency of the plurality of high voltage pulses.

15. The method of claim 10, further comprising a step of manipulating the distal tip portion to steer the catheter system to a second treatment location within the calcified heart valve.

16. The method of claim 10, further comprising the steps of:
at least partially deflating the balloon;
moving the catheter system within the patient’s heart;
manipulating the distal tip portion to steer the catheter system to a second treatment location within the calcified heart valve.

17. A method of treatment and repair of a calcified heart valve of a patient with a heart valve catheter system having a steerable distal tip portion, the method comprising:
inserting an inflatable balloon of the catheter system into a patient’s heart and moving the inflatable balloon to the calcified heart valve, the catheter system comprising a steerable distal tip portion that extends distally beyond a distal end of the inflatable balloon, the catheter system further including at least two electrodes located inside the balloon to form a first emitter, the at least two electrodes connected at the proximal portion of the catheter system to a high voltage pulse generator to generate a high voltage pulse;
inflating the balloon to less than a size of an inner area of the calcified heart valve;
deflecting the distal tip portion to a first treatment location so that a portion of an exterior surface of the balloon is in contact with one or more leaflets of the heart valve; and
providing a high voltage pulse from the high voltage pulse generator to create a spark from the first emitter for creating an energy wave for propagation through the fluid within the balloon to provide force transmitted through the balloon to break up any calcification at the first treatment location.

18. The method of claim 17, further comprising steps of manipulating the heart valve catheter system to a second treatment location by steering the distal tip portion inside the heart without deflating the balloon, and then providing a high voltage pulse from the high voltage pulse generator to create a spark from an emitter for creating an energy wave for propagation through the fluid within the balloon to provide force transmitted through the balloon to break up the calcifications at the second treatment location.

19. The method of claim 17, wherein the step of inserting the inflatable balloon into the heart includes inserting the inflatable balloon in a deflated state and inflating the balloon once the balloon is positioned in the interior of the heart.

20. The method of claim of claim 17, wherein the step of inflating the balloon to less than the size of the inner area of the heart valve comprises inflating the balloon to less than 80 percent of the size of the inner area of the heart valve.

21. The method of claim 17, wherein the at least two electrodes comprise six electrodes that form the first emitter, a second emitter, and a third emitter, and further comprising providing a high voltage pulse from the high voltage pulse generator to create a spark from the second and third emitters for creating at least one other energy wave for propagation through the fluid within the balloon to provide force to break up the calcification at the first treatment location.

22. The method of claim 21, wherein the six electrodes are arranged in pairs that are spaced from each other by a distance that is smaller than the distance between adjacent pairs of electrodes.

23. An elongate catheter system for providing high energy acoustic waves to a heart valve region of a subject, the catheter system comprising:
an inflatable balloon at a distal portion of the catheter system, the inflatable balloon adapted to:
traverse a vessel of the subject in a radial retracted configuration to reach a treatment site in the heart valve region; and
receive fluid to inflate to an expanded configuration at the treatment site;
a deflectable distal tip portion distal to the inflatable balloon;
an emitter within the inflatable balloon;
a conductive pathway for supplying energy to the emitter to generate high energy acoustic waves within fluid inflating the balloon;
a movable control member operatively connected to the distal portion for deflecting the distal tip portion in a direction away from the longitudinal axis of the catheter system;
a pulse generator for creating a spark from the emitter to create an energy wave for propagation through fluid within the balloon and provide force to break up calcifications; and
a control module for controlling the pulse generator.
